SEQUENCE LISTING (17 sequences)

SEQ ID NO: 1 - RNA, 103 nt, synthetic construct
  source 1..103: mol_type = other RNA
  modified_base 25: mod_base = OTHER; note = Uridine is tagged with tetrazine (Tz1)
gggcgcttgtttcggcgtgggtagttttagagctagacatagcaagttaaaataaggctagtccgttatcaacttgaaaaagtggcaccgagtcggtgctttt

SEQ ID NO: 2 - RNA, 103 nt, synthetic construct; identical to SEQ ID NO: 1
  source 1..103: mol_type = other RNA
  modified_base 28: mod_base = OTHER; note = Uridine is tagged with tetrazine (Tz1)

SEQ ID NO: 3 - RNA, 103 nt, synthetic construct; identical to SEQ ID NO: 1
  source 1..103: mol_type = other RNA
  modified_base 25: mod_base = OTHER; note = Uridine is tagged with tetrazine (Tz2)

SEQ ID NO: 4 - RNA, 103 nt, synthetic construct; identical to SEQ ID NO: 1
  source 1..103: mol_type = other RNA
  modified_base 28: mod_base = OTHER; note = Uridine is tagged with tetrazine (Tz2)

SEQ ID NO: 5 - RNA, 101 nt, synthetic construct
  source 1..101: mol_type = other RNA
  modified_base 22: mod_base = OTHER; note = Uridine is tagged with tetrazine (Tz1)
gggcgaggagctgttcaccggttttagagctagaaatagcaagttaaaataaggctagtccgttatcaacttgaaaaagtggcaccgagtcggtgcttttt

SEQ ID NO: 6 - RNA, 101 nt, synthetic construct; identical to SEQ ID NO: 5
  source 1..101: mol_type = other RNA
  modified_base 25: mod_base = OTHER; note = Uridine is tagged with tetrazine (Tz1)

SEQ ID NO: 7 - RNA, 101 nt, synthetic construct; identical to SEQ ID NO: 5
  source 1..101: mol_type = other RNA
  modified_base 22: mod_base = OTHER; note = Uridine is tagged with tetrazine (Tz2)

SEQ ID NO: 8 - RNA, 101 nt, synthetic construct; identical to SEQ ID NO: 5
  source 1..101: mol_type = other RNA
  modified_base 25: mod_base = OTHER; note = Uridine is tagged with tetrazine (Tz2)

SEQ ID NO: 9 - RNA, 101 nt, synthetic construct; identical to SEQ ID NO: 5
  source 1..101: mol_type = other RNA
  modified_base order(28..42,46,48,50,52..57,60..61,66..67,70..81,83..101): mod_base = OTHER; note = 2-Prime-O-methyl ribonucleotides

SEQ ID NO: 10 - RNA, 101 nt, synthetic construct; identical to SEQ ID NO: 5
  source 1..101: mol_type = other RNA
  modified_base 25: mod_base = OTHER; note = Uridine is tagged with tetrazine (Tz1)
  modified_base order(28..42,46,48,50,52..57,60..61,66..67,70..81,83..101): mod_base = OTHER; note = 2-Prime-O-methyl ribonucleotides

SEQ ID NO: 11 - RNA, 101 nt, synthetic construct; identical to SEQ ID NO: 5
  source 1..101: mol_type = other RNA
  modified_base 25: mod_base = OTHER; note = Uridine is tagged with tetrazine (Tz2)
  modified_base order(28..42,46,48,50,52..57,60..61,66..67,70..81,83..101): mod_base = OTHER; note = 2-Prime-O-methyl ribonucleotides

SEQ ID NO: 12 - RNA, 101 nt, synthetic construct
  source 1..101: mol_type = other RNA
  modified_base order(28..42,46,48,50,52..57,60..61,66..67,70..81,83..101): mod_base = OTHER; note = 2-Prime-O-methyl ribonucleotides
ggtgagtgagtgtgtgcgtggttttagagctagaaatagcaagttaaaataaggctagtccgttatcaacttgaaaaagtggcaccgagtcggtgcttttt

SEQ ID NO: 13 - RNA, 101 nt, synthetic construct; identical to SEQ ID NO: 12
  source 1..101: mol_type = other RNA
  modified_base 25: mod_base = OTHER; note = Uridine is tagged with tetrazine (Tz1)
  modified_base order(28..42,46,48,50,52..57,60..61,66..67,70..81,83..101): mod_base = OTHER; note = 2-Prime-O-methyl ribonucleotides

SEQ ID NO: 14 - RNA, 101 nt, synthetic construct; identical to SEQ ID NO: 12
  source 1..101: mol_type = other RNA
  modified_base 25: mod_base = OTHER; note = Uridine is tagged with tetrazine (Tz2)
  modified_base order(28..42,46,48,50,52..57,60..61,66..67,70..81,83..101): mod_base = OTHER; note = 2-Prime-O-methyl ribonucleotides

SEQ ID NO: 15 - RNA, 101 nt, synthetic construct; identical to SEQ ID NO: 5
  source 1..101: mol_type = other RNA
  modified_base order(28..42,46,48,50,52..57,60..61,66..67,70..81,83..101): mod_base = OTHER; note = 2-Prime-O-methyl ribonucleotides
  modified_base 25: mod_base = OTHER; note = Uridine is tagged with either tetrazine (Tz1) or tetrazine (Tz2)

SEQ ID NO: 16 - protein, 5 aa, synthetic construct
RRWQW

SEQ ID NO: 17 - protein, 5 aa, synthetic construct
RLRWR

(19) United States
(12) Patent Application Publication
(10) Pub. No.: US 2026/0062702 A1
(43) Pub. Date: Mar. 5, 2026

(54) TEMPORAL CONTROL OF CRISPR-Cas9 ACTIVITY USING BIOORTHOGONAL CHEMISTRY

(71) Applicant: The Research Foundation for the State University of New York, Albany, NY (US)

(72) Inventors: Maksim Royzen, Albany, NY (US); Bhoomika Pandit, Albany, NY (US)

(21) Appl. No.: 19/316,343

(22) Filed: Sep. 2, 2025

Related U.S. Application Data
(60) Provisional application No. 63/689,090, filed on Aug. 30, 2024.

(51) Int. Cl. C12N 15/113; C12N 9/22; C12N 15/11; C12N 15/90

(52) U.S. Cl. CPC C12N 15/113; C12N 9/226; C12N 15/11; C12N 15/907; C12N 2310/20; C12N 2310/3181

(57) ABSTRACT

Disclosed are systems and methods for reversible, chemically inducible regulation of CRISPR-Cas9 activity using bioorthogonal chemistry. The invention includes modified guide RNAs (sgRNAs) incorporating a first bioorthogonal reactive group that does not interfere with native Cas9 function. Upon introduction of a CRISPR suppressor comprising a second complementary bioorthogonal reactive group, a rapid and selective chemical reaction occurs under physiological conditions, resulting in inhibition of Cas9-mediated genome editing. The suppressors may include small molecules, peptides, or nucleic acid analogs designed to sterically or conformationally disrupt the ribonucleoprotein complex. This platform enables precise spatial and temporal control over CRISPR-Cas9 activity, with potential applications in improving editing specificity and reducing off-target effects. The disclosed methods are modular, biocompatible, and adaptable to a variety of gene editing contexts.

### CROSS-REFERENCE TO RELATED APPLICATIONS

This application claims the benefit of U.S. Provisional Application No. 63/689,090, filed on Aug. 30, 2024, which is incorporated herein by reference in its entirety.

### SEQUENCE LISTING

The instant application contains a Sequence Listing which has been submitted via Patent Center and is hereby incorporated by reference in its entirety. Said .xml copy, created on Sep. 1, 2025, is named 010-24-37US01_SEQ, and is 28,961 bytes in size.

### BACKGROUND OF THE INVENTION

### Field of the Invention

The CRISPR (clustered regularly interspaced short palindromic repeats)-Cas9 system has become a widely popular tool for genome engineering in different organisms and biological systems. The most frequently used system is the type II Cas9 from Streptococcus pyogenes strain SF370 (SpyCas9) and single guide RNA (sgRNA), which targets specific DNA sequences in the genome to create a blunt-ended double-strand breaks. In recent years, CRISPR technology has been evaluated in several human clinical trials to treat various cancers, eye disease and chronic infection. Perhaps the most striking biomedical breakthrough has been in the treatment of sickle cell disease, the most common inherited hemoglobinopathy worldwide. In 2023, FDA approve two milestone sickle cell disease treatments, Casgevy and Lyfgenia, that are based on CRISPR technology.

One of the biggest hurdles in the development of new CRISPR-Cas9-based drugs is elimination of the so-called off-target effects. After administration, sgRNA-Cas9 ribonuclear protein will stay in the body for a long time. In addition to the intended target gene, CRISPR-Cas9-induced indels can occur at unintended off-target cleavage sites that have as many as five mismatches within the protospacer. Editing of off-target sites is known to occur on a slower timescale than the on-target sites. Therefore, there is considerable interest in developing systems that allow temporal control CRISPR-Cas9 nuclease activity. Such systems will inactivate either Cas9 or sgRNA once the target gene has been edited to prevent further editing of off-target sites.

A number of bio-engineering approaches have already been reported to modulate activity of Cas9. For example, Choudhary group fused Cas9 to the destabilized polypeptide domains of E. coli's dihydrofolate reductase. These domains are unstable and rapidly target the fusion protein for proteasomal degradation. The destabilized domains can be stabilized upon addition of trimethoprim compound, which prevents proteasomal degradation. Choudhary group have shown that by controlling the concentration and time of administration of trimethoprim, both the dosage and temporal control of Cas9 activity can be achieved. Alternative methods to modulate Cas9 activity have been reported by Bondy-Denomy and Yee groups.

### BRIEF SUMMARY OF THE INVENTION

Embodiments disclosed herein are directed to methods, compositions, and kits for temporally controlling CRISPR-Cas9 nuclease activity in a cell through the use of bioorthogonal chemistry. In some embodiments, a method is provided that includes introducing into a cell a ribonucleoprotein (RNP) complex comprising a single guide RNA (sgRNA) and a Cas9 protein, wherein the sgRNA is covalently tagged with a first bioorthogonal reactive group at a nucleotide within the repeat region of the sgRNA. A second composition comprising a small molecule CRISPR suppressor with a second bioorthogonal reactive group is subsequently administered to the cell. The first and second reactive groups are members of a bioorthogonal click chemistry pair that react under physiological conditions to form a covalent adduct, thereby inhibiting CRISPR-Cas9 nuclease activity.

In certain embodiments, the RNP complex is formed prior to cellular introduction, allowing for precise temporal control of genome editing activity through delayed administration of the suppressor. The covalent adduct formed between the tagged sgRNA and the suppressor may sterically hinder the sgRNA-Cas9 interaction, thereby deactivating the complex.

Additional embodiments are directed to kit-of-parts compositions comprising a first component including a Cas9 protein and a single guide RNA comprising a first bioorthogonal reactive group covalently attached to a nucleotide within the repeat region of the sgRNA, and a second component comprising a small molecule CRISPR suppressor comprising a second bioorthogonal reactive group. In some embodiments, these two components are housed in physically separate compartments of a single package. The reactive groups are capable of undergoing a bioorthogonal click reaction under physiological conditions to inhibit CRISPR-Cas9 nuclease activity.

Further embodiments are directed to compositions comprising a Cas9 protein and a single guide RNA covalently linked to a small molecule CRISPR suppressor through a bioorthogonal click chemistry reaction product. The covalent adduct is formed by reaction of a first reactive group covalently attached to a nucleotide within the sgRNA and a second reactive group on the small molecule suppressor, and this adduct inhibits CRISPR-Cas9 nuclease activity. In some cases, the adduct is formed via an inverse electron demand Diels-Alder cycloaddition.

Various embodiments also disclose specific configurations of the reactive groups. For example, the first reactive group may be a tetrazine and the second reactive group may be a trans-cyclooctene. Alternatively, the reactive pair may comprise a cyclooctyne and an azide. The sgRNA may be tagged specifically at a uridine nucleotide within the repeat:anti-repeat region and may include chemical stabilizations such as 2′-O-methyl, 2′-fluoro modifications, or phosphorothioate backbone.

The small molecule CRISPR suppressor may include a cell-penetrating peptide, a peptide nucleic acid (PNA), a synthetic oligonucleotide analog, or other sterically hindering moieties. In certain embodiments, the suppressor has a molecular weight under about 1200 Da. The timing of covalent bond formation between the sgRNA and the suppressor may be controlled, occurring after a defined period post-transfection to enable temporal regulation of gene editing activity.

Other embodiments relate to compositions in which the small molecule CRISPR suppressor includes a cell-permeable moiety covalently linked to a second bioorthogonal reactive group selected from the group consisting of trans-cyclooctene, cyclooctyne, alkyne, azide, and hydrazine. In some embodiments, the second reactive group is trans-cyclooctene. In particular examples, the small molecule CRISPR suppressor comprises a trans-cyclooctene-modified peptide nucleic acid selected from the group consisting of AAA-PNA-TCO and TTT-PNA-TCO and is formulated for sequential administration with a tetrazine-modified sgRNA.

Still further embodiments are directed to methods wherein the second composition comprises such suppressor compositions, and wherein the second composition is administered sequentially after introduction of the ribonucleoprotein complex to the cell in order to enable temporal control of Cas9 activity.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

FIG. 1 presents a schematic representation of controlled turn-off of CRISPR-Cas9 activity using bioorthogonal chemistry between TCO and Tz.

FIG. 2A presents the incorporation of Tz into the repeat region of sgRNA. The important uridine residues are numbered U₁-U₄. FIG. 2B presents phosphoramidites and Tz-NHS ester that were used to tag sgRNAs with Tz.

FIG. 3A presents solvent accessible surface area of the RNA nucleotides in the protein:RNA:DNA complex (PDB ID: 4008). FIG. 3B presents atomic contacts within 4 Å of the four Us of the repeat sequence with Cas9, in the sgRNA-Cas9 complex.

FIG. 3C presents structural context of the four Us in the sgRNA-Cas9 complex. The potential modification sites (atoms C5 and 02′) are shown as spheres. sgRNA is shown in gray and Cas9 is shown in brown. FIG. 3D presents Tz₁ and Tz₂ modifications modeled on U₁ and FIG. 3E presents Tz₁ and Tz₂ modifications modeled on U₄.

FIG. 4A presents an analysis of CRISPR-Cas9 experiments using agarose gel electrophoresis wherein, Lane 1: DNA ladder; Lane 2: linearized pBR322 plasmid; Lane 3: linearized pBR322 plasmid and sgRNA1; Lane 4: linearized pBR322 plasmid and sgRNA1-(U₁Tz₁); Lane 5: linearized pBR322 plasmid and sgRNA1-(U₄Tz₁); Lane 6: linearized pBR322 plasmid and sgRNA1-(U₁Tz₂); Lane 7: linearized pBR322 plasmid and sgRNA1-(U₄Tz₂). FIG. 4B presents a bar chart representing the percentage of cut DNA upon treatment with the constructs in part FIG. 4A. Lane numbering is the same as in FIG. 4A. All CRISPR experiments were performed in duplicate. Error bars represent±s.d.

FIG. 5A presents an analysis of CRISPR-Cas9 experiments using agarose gel electrophoresis wherein, Lane 1: DNA ladder; Lane 2: linearized eGFP-N1 plasmid; Lane 3: linearized eGFP-N1 plasmid and sgRNA2; Lane 4: linearized eGFP-N1 plasmid and sgRNA2-(U₁Tz₁); Lane 5: linearized eGFP-N1 plasmid and sgRNA2-(U₄Tz₁); Lane 6: linearized eGFP-N1 plasmid and sgRNA2-(U₁Tz₂); Lane 7: linearized eGFP-N1 plasmid and sgRNA2-(U₄Tz₂). FIG. 5B presents a bar chart representing the percentage of cut DNA upon treatment with the constructs in FIG. 5A. Lane numbering is the same as in FIG. 5A. All CRISPR experiments were performed in duplicate. Error bars represent ±s.d.

FIG. 6A and FIG. 6B present a library of small molecule TCO-modified CRISPR suppressors that are based on FIG. 6A PNA and FIG. 6B CPP.

FIG. 7A presents an analysis of CRISPR-Cas9 experiments in the presence of TCO-modified CRISPR suppressors wherein, Lane 1: linearized pBR322 plasmid; Lane 2: linearized pBR322 plasmid and sgRNA1-(U₄Tz₂); Lane 3: linearized pBR322 plasmid, sgRNA1-(U₄Tz₂) and TCO-CPP-RRWQW (SEQ ID NO: 16); Lane 4: linearized pBR322 plasmid, sgRNA1-(U₄Tz₂) and TCO-CPP-RLRWR (SEQ ID NO: 17); Lane 5: linearized pBR322 plasmid, sgRNA1-(U₄Tz₂) and TTT-PNA-TCO; Lane 6: linearized pBR322 plasmid, sgRNA1-(U₄Tz₂) and AAA-PNA-TCO; Lane 7: linearized pBR322 plasmid and sgRNA1-(U₄Tz₁); Lane 8: linearized pBR322 plasmid, sgRNA1-(U₄Tz₁) and TCO-CPP-RRWQW (SEQ ID NO: 16); Lane 9: linearized pBR322 plasmid, sgRNA1-(U₄Tz₁) and TCO-CPP-RLRWR (SEQ ID NO: 17); Lane 10: linearized pBR322 plasmid, sgRNA1-(U₄Tz₁) and TTT-PNA-TCO; Lane 11: linearized pBR322 plasmid, sgRNA1-(U₄Tz₁) and AAA-PNA-TCO. FIG. 7B presents a bar chart representing the percentage of cut DNA upon treatment with the constructs in FIG. 7A. Lane numbering is the same as in FIG. 7A. All CRISPR experiments were performed in duplicate. Error bars represent ±s.d.

FIG. 8A presents an analysis of CRISPR-Cas9 experiments in the presence of TCO-modified CRISPR suppressors wherein, Lane 1: linearized eGFP-N1 plasmid; Lane 2: linearized eGFP-N1 plasmid and sgRNA2-(U₄Tz₁); Lane 3: linearized eGFP-N1 plasmid, sgRNA2-(U₄Tz₁) and TCO-CPP-RRWQW (SEQ ID NO: 16); Lane 4: linearized eGFP-N1 plasmid, sgRNA2-(U₄Tz₁) and TCO-CPP-RLRWR (SEQ ID NO: 17); Lane 5: linearized eGFP-N1 plasmid, sgRNA2-(U₄Tz₁) and TTT-PNA-TCO; Lane 6: linearized eGFP-N1 plasmid, sgRNA2-(U₄Tz₁) and AAA-PNA-TCO; Lane 7: linearized eGFP-N1 plasmid and sgRNA2-(U₄Tz₂); Lane 8: linearized eGFP-N1 plasmid, sgRNA2-(U₄Tz₂) and TCO-CPP-RRWQW (SEQ ID NO: 16); Lane 9: linearized eGFP-N1 plasmid, sgRNA2-(U₄Tz₂) and TCO-CPP-RLRWR (SEQ ID NO: 17); Lane 10: linearized eGFP-N1 plasmid, sgRNA2-(U₄Tz₂) and TTT-PNA-TCO; Lane 11: linearized eGFP-N1 plasmid, sgRNA2-(U₄Tz₂) and AAA-PNA-TCO. FIG. 8B presents a bar chart representing the percentage of cut DNA upon treatment with the constructs in FIG. 8A. Lane numbering is the same as in FIG. 8A. All CRISPR experiments were performed in duplicate. Error bars represent ±s.d.

FIG. 9A-FIG. 9T present an assessment of cell permeability of TCO-modified CRISPR suppressors wherein, HEK293 cells were treated with OG-Tz alone (FIG. 9A-FIG. 9D), or in combination with AAA-PNA-TCO (FIG. 9E-FIG. 9H), TTT-PNA-TCO (FIG. 9I-FIG. 9L), TCO-CPP-RRWQW (SEQ ID NO: 16) (FIG. 9M-FIG. 9P) and TCO-CPP-RLRWR (SEQ ID NO: 17) (FIG. 9Q-FIG. 9T).

FIG. 10A-FIG. 10E present flow cytometry of GFP-expressing HEK293 cells treated with sgRNA 3-(U₄Tz₁) and TCO-modified CRISPR suppressors. Histograms of untreated cells are shown in black. The cells treated with sgRNA 3-(U₄Tz₁) are shown in green. The cells treated with sgRNA 3-(U₄Tz₁) and TCO-modified CRISPR suppressors are shown in blue. FIG. 10A presents an overlay of histograms of the untreated cells, cells treated with sgRNA 3-(U₄Tz₁) and cells treated with sgRNA 3-(U₄Tz₁) and TCO-CPP-RRWQW (SEQ ID NO: 16); FIG. 10B presents an overlay of histograms of the untreated cells, cells treated with sgRNA 3-(U₄Tz₁) and cells treated with sgRNA 3-(U₄Tz₁) and TCO-CPP-RLRWR (SEQ ID NO: 17); FIG. 10 C presents an overlay of histograms of the untreated cells, cells treated with sgRNA3-(U₄Tz₁) and cells treated with sgRNA 3-(U₄Tz₁) and TTT-PNA-TCO; FIG. 10D presents an overlay of histograms of the untreated cells, cells treated with sgRNA 3-(U₄Tz₁) and cells treated with sgRNA 3-(U₄Tz₁) and AAA-PNA-TCO; and FIG. 10E presents the decrease of MFI of GFP relative to the untreated GFP-expressing HEK293 cells. All experiments were performed in duplicate. Error bars represent ±s.d.

FIG. 11A-FIG. 11E present flow cytometry of GFP-expressing HEK293 cells treated with sgRNA 3-(U₄Tz₂) and TCO-modified CRISPR suppressors. Histograms of untreated cells are shown in black. The cells treated with sgRNA 3-(U₄Tz₂) are shown in green. The cells treated with sgRNA 3-(U₄Tz₂) and TCO-modified CRISPR suppressors are shown in blue. FIG. 11A presents an overlay of histograms of the untreated cells, cells treated with sgRNA 3-(U₄Tz₂) and cells treated with sgRNA 3-(U₄Tz₂) and TCO-CPP-RRWQW (SEQ ID NO: 16); FIG. 11B presents an overlay of histograms of the untreated cells, cells treated with sgRNA 3-(U₄Tz₂) and cells treated with sgRNA 3-(U₄Tz₂) and TCO-CPP-RLRWR (SEQ ID NO: 17); FIG. 11C presents an overlay of histograms of the untreated cells, cells treated with sgRNA 3-(U₄Tz₂) and cells treated with sgRNA 3-(U₄Tz₂) and TTT-PNA-TCO; FIG. 11D presents an overlay of histograms of the untreated cells, cells treated with sgRNA 3-(U₄Tz₂) and cells treated with sgRNA 3-(U₄Tz₂) and AAA-PNA-TCO; FIG. 11E presents the decrease of MFI of GFP relative to the untreated GFP-expressing HEK293 cells. All experiments were performed in duplicate. Error bars represent ±s.d.

FIG. 12 presents a Western blot analysis of CRISPR-Cas9 experiments targeting VEGFA. Cells were treated with: Lane 1: sgRNA4-(U₄Tz₂) and TCO-CPP-RLRWR (SEQ ID NO: 17); Lane 2: sgRNA4-(U₄Tz₂) and TCO-CPP-RRWQW (SEQ ID NO: 16); Lane 3: sgRNA4-(U₄Tz₂); Lane 4: linearized eGFP-N1 plasmid, sgRNA2-(U₄Tz₁) and TCO-CPP-RLRWR (SEQ ID NO: 17); Lane 5: sgRNA4-(U₄Tz₁) and TCO-CPP-RLRWR (SEQ ID NO: 17); Lane 6: sgRNA4-(U₄Tz₁) and TCO-CPP-RRWQW (SEQ ID NO: 16); Lane 7: sgRNA4-(U₄Tz₁); Lane 8: untreated cells. a-tubulin was used as a loading control.

FIG. 13A presents PAGE analysis of purified sgRNAs: Lane 1: sgRNA1-(U₄Tz₂); Lane 2: sgRNA1-(U₁Tz₂); Lane 3: sgRNA1-(U₄Tz₁); Lane 4: sgRNA1-(U₁Tz₁); Lane 5: reference. FIG. 13B presents deconvoluted ESI-MS analysis of sgRNA1-(U₁Tz₂); and FIG. 13C presents deconvoluted ESI-MS analysis of sgRNA1-(U₄Tz₂).

FIG. 14 presents Fluorescence spectra of OG-Tz (Iₑₓ=490 nm) by itself and conjugated to different TCO-modified CRISPR suppressors.

FIG. 15A-15D present flow cytometry of HEK293 cells treated with OG-Tz alone (black) and with OG-Tz and TCO-modified CRISPR suppressors (green). FIG. 15A presents TTT-PNA-TCO; FIG. 15B presents TCO-CPP-RRWQW (SEQ ID NO: 16); FIG. 15C presents TCO-CPP-RLRWR (SEQ ID NO: 17). FIG. 15D increase of MFI of OG-Tz fluorescence after addition of TTT-PNA-TCO, TCO-CPP-RRWQW (SEQ ID NO: 16), TCO-CPP-RLRWR (SEQ ID NO: 17). All experiments were performed in duplicate. Error bars represent ±s.d.

FIG. 16 presents an analysis of CRISPR-Cas9 experiments using agarose gel electrophoresis. Lane 1: linearized eGFP-N1 plasmid; Lane 2: linearized eGFP-N1 plasmid and sgRNA3-(U₄Tz₁); Lane 3: linearized eGFP-N1 plasmid and sgRNA3-(U₄Tz₂).

FIG. 17 presents PAGE analysis of purified sgRNAs: Lane 1: sgRNA2-(U₄Tz₂); Lane 2: sgRNA2-(U₁Tz₂); Lane 3: sgRNA2-(U₄Tz₁); Lane 4: sgRNA2-(U₁Tz₁); Lane 5: unmodified sgRNA2; Lane 6: reference.

FIG. 18 presents tetrazines and trans-cyclooctenes substituted with different known functional groups and linkers that are useful in the bioorthogonal reactions disclosed herein.

FIG. 19 presents examples of substituted trans-cyclooctenes, substituted norbornenes, and substituted cyclopropenes that are useful in the bioorthogonal reactions disclosed herein.

### DETAILED DESCRIPTION OF THE INVENTION

Described herein is a platform for controlling CRISPR-Cas9 activity based on the covalent labeling of single-guide RNA (sgRNA) with a bioorthogonal reactive group and subsequent inactivation of the CRISPR ribonucleoprotein (RNP) complex through reaction with a small molecule or peptide containing a complementary reactive group. This reaction occurs selectively and rapidly under physiological conditions without perturbing native cellular functions.

The bioorthogonal reaction may be selected from a range of known, biocompatible click chemistry reactions, including but not limited to inverse-electron-demand Diels-Alder reactions, strain-promoted azide-alkyne cycloadditions (SPAAC), strain-promoted alkyne-nitrone cycloadditions (SPANC), isocyanide [4+1] click reaction, Staudinger reaction, UV-induced 1,3-dipolar cycloaddition reaction between tetrazoles and vinyl compounds and oxime ligation.

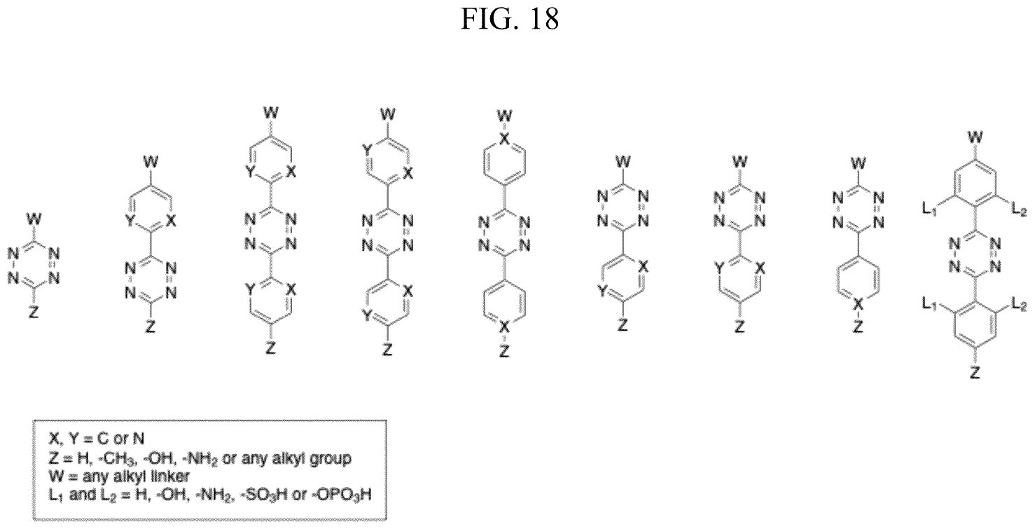

Tetrazines and trans-cyclooctenes can be substituted with different known functional groups and linkers shown in FIG. 18.

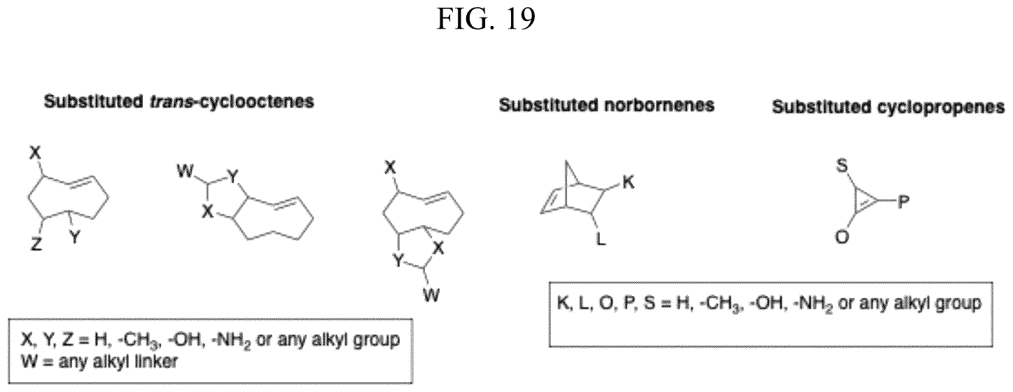

Other strained alkenes, such as norbornenes and cyclopropenes, shown in FIG. 19 can be used instead of trans-cyclooctene. They are also known to react with tetrazine via inverse-electron-demand Diels-Alder reaction.

In a preferred embodiment, a tetrazine (Tz) moiety is conjugated to a uridine in the repeat:anti-repeat region of sgRNA, and a trans-cyclooctene (TCO)-modified small molecule or peptide serves as the suppressor. However, in alternative embodiments, the invention contemplates the use of functionally equivalent chemical groups with comparable reactivity profiles. For example, TCO and Tz groups can be used in reverse: TCO is conjugated to a uridine in the repeat:anti-repeat region of sgRNA, and a Tz-modified small molecule or peptide serves as the suppressor. Alternatively, to employ SPAAC chemistry, cyclooctyne is conjugated to a uridine in the repeat:anti-repeat region of sgRNA, and an azide-modified small molecule or peptide serves as the suppressor.

The CRISPR-Cas9 system has become a widely used tool for genome engineering. Here we disclose, inter alia, a new method for small-molecule control of CRISPR-Cas9 using bioorthogonal chemistry between tetrazine (Tz) and trans-cyclooctene (TCO). We utilized molecular modeling studies and identified a unique position on single guide RNA (sgRNA) that can be site-specifically tagged with Tz without disrupting its activity. We also synthesized a series of TCO-modified CRISPR suppressors. When exogenously added, they click to the Tz-tagged sgRNA, perturb the system and drastically reduce the nuclease activity. The most successful suppressor is a TCO-modified six amino acid long cell-penetrating peptide, which shows excellent cell permeability. We showed that our method to control CRISPR-Cas9 nuclease activity is general by applying it to three different sgRNAs. We also showed that our method works in solution, as well as live HEL293 cells. We utilized flow cytometry to demonstrate temporal control of CRISPR-Cas9 targeting GFP. Lastly, we showed the therapeutic potential of our method by targeting vascular endothelial growth factor A (VEGFA).

The present embodiments disclosed herein relate to methods and compositions for modulating the activity of CRISPR-Cas9 gene editing systems through chemically inducible control using bioorthogonal reactive groups. More specifically, according to certain embodiments there are provided systems for temporal and spatial control of CRISPR-Cas9 nuclease activity via selective chemical modification of sgRNA and small-molecule CRISPR suppressors utilizing inverse electron demand Diels-Alder (IEDDA) or other biocompatible click reactions.

The ability to precisely edit the genome has revolutionized biological research and therapeutic development. However, one of the major challenges in clinical translation of CRISPR-Cas9 systems is managing off-target editing that may occur over time, especially when CRISPR components persist in cells. Therefore, strategies enabling tight temporal control over CRISPR-Cas9 activity are desirable. Previous efforts to regulate Cas9 activity have relied on protein engineering, chemical degradation tags, or inducible transcription systems. These approaches, while useful, often involve large genetic constructs or irreversible modifications. The present invention addresses this challenge by enabling reversible, small-molecule-based control of CRISPR activity via bioorthogonal chemical reactions.

As used herein the term bioorthogonal reactive group is a chemical moiety that undergoes a highly selective chemical reaction under physiological conditions without interfering with native biological functions. A “bioorthogonal reactive group” refers to a functional group capable of undergoing a specific, covalent reaction with a corresponding partner in a living system without cross-reactivity with endogenous biological nucleophiles. Example pairs include tetrazine/TCO (via inverse electron demand Diels-Alder) and azide/cyclooctyne (via strain-promoted azide-alkyne cycloaddition).

As used herein the term IEDDA reaction is an inverse-electron-demand Diels-Alder reaction between an electron-deficient diene (e.g., tetrazine) and an electron-rich dienophile (e.g., TCO or norbornene).

As used herein the term CRISPR suppressor is a compound that modulates CRISPR activity by reacting with a tagged sgRNA to disrupt its function. In addition, the “CRISPR suppressor” refers to a small molecule, peptide, nucleic acid analog, or other synthetic moiety that is capable of covalently reacting with a modified sgRNA and subsequently disrupting or inhibiting the activity of the sgRNA-Cas9 complex. In one aspect, this inhibition is due to steric hindrance, preventing proper sgRNA-Cas9 assembly or target DNA binding.

As used herein, “inhibits CRISPR-Cas9 nuclease activity” means that the DNA cleavage activity of Cas9 at a targeted genomic locus is reduced or abrogated following the formation of a covalent adduct between the sgRNA and the suppressor molecule. In some embodiments, the inhibition is steric in nature, while in others it may affect RNA folding, Cas9 binding, or target DNA recognition.

As used herein equivalents are any functional group, compound, or method that performs substantially the same function in substantially the same way to achieve substantially the same result as the claimed element.

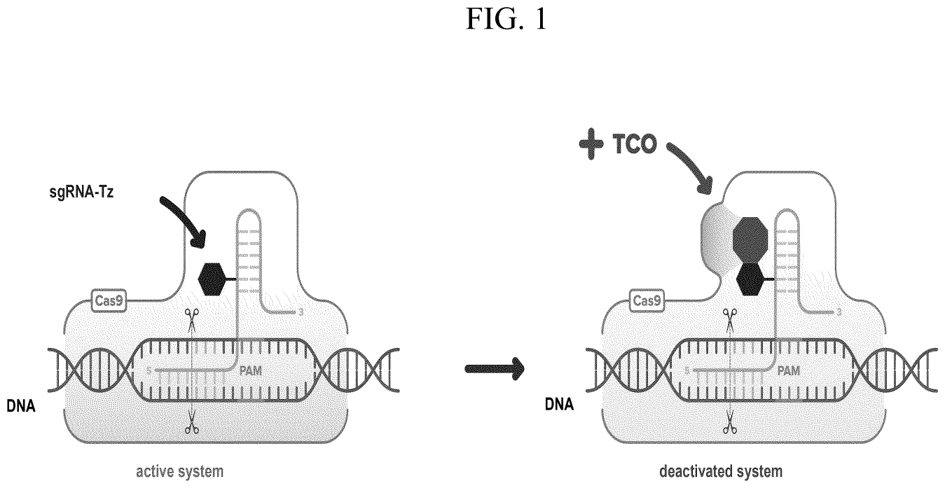

Certain embodiments disclosed herein are directed to methods and compositions for controlling CRISPR-Cas9 activity using small molecule bioorthogonal chemistry between trans-cyclooctene (TCO) and tetrazine (Tz). The reaction between TCO and Tz has exceptionally fast kinetics. The two reacting groups are biocompatible and have high potential for bio-medical translation. The design is schematically illustrated in FIG. 1. Specific embodiments disclosed herein are directed to modification of sgRNA with a covalently attached Tz tag. Tz tagging of a single nucleotide has been experimentally optimized to cause minimal perturbation to sgRNA's function in Cas9-assisted nuclease activity. Thus, the construct shown on the left in FIG. 1 is constitutively functional. To control CRISPR activity, TCO-containing small molecule suppressor will be exogenously added. The TCO group of the suppressor will react via bioorthogonal click reaction with the Tz group attached to sgRNA. The conjugation product will cause perturbation in the domain of sgRNA/Cas9 complex that is important for nuclease activity, thus rendering the ribonucleoprotein (RNP) complex inactive. In comparison to already reported methods to control CRISPR-Cas9 activity, the key advantage of the design disclosed herein is that it involves a small molecule RNA tag, which minimally perturbs sgRNA's structure, and small molecule suppressors which can be easily administered to control the nuclease activity.

The embodiments herein are not limited to a specific reactive pair. The term “first bioorthogonal reactive group” refers to any moiety covalently attached to the sgRNA that can undergo a chemoselective, high-rate reaction under physiological conditions with a complementary “second bioorthogonal reactive group” on a small molecule or peptide. Examples of suitable first bioorthogonal reactive groups (installed on sgRNA) include, but are not limited to: Tetrazines, Azides, Cyclopropenes, Vinylboronic acids, and Alkynes (e.g., terminal alkynes, strained alkynes). Examples of suitable second bioorthogonal reactive groups (on the CRISPR suppressor) include: Trans-cyclooctene (TCO), Norbornene, Bicyclononyne (BCN), DBCO (dibenzocyclooctyne), and Cyclooctyne.

The reaction between the first and second bioorthogonal reactive groups must proceed with high rate constants (>1,000 M⁻¹s⁻¹ preferred) and exhibit selectivity under biological conditions.

In some embodiments, these reactions may include: Tetrazine+TCO (inverse-electron-demand Diels-Alder), Azide+DBCO (SPAAC), Cyclopropene+tetrazine (IEDDA), Nitrone+alkyne (SPANC)

The sgRNA is modified at a structurally permissive site within the repeat:anti-repeat region, preferably at uridine U₄, using chemical conjugation methods compatible with solid-phase synthesis or post-synthetic labeling. The bioorthogonal group may be installed through: Modified nucleoside phosphoramidites; NHS-ester or click-conjugation chemistry; Enzymatic or ribozyme-mediated site-specific ligation. The modification is selected so as not to interfere with CRISPR-Cas9 activity prior to reaction with the CRISPR suppressor.

The suppressor molecule comprises: A second bioorthogonal reactive group that reacts selectively with the group on sgRNA; and a steric, conformational, electrostatic, or base-pairing disrupting domain that perturbs the sgRNA:Cas9 interaction or sgRNA structure upon conjugation. The suppressor may be: a peptide nucleic acid (PNA), a small cell-penetrating peptide (CPP), a small molecule with RNA binding or sterically hindering features. Preferably, the suppressor is cell-permeable, has a molecular weight under 1500 Da, and is stable in biological media.

The system can be applied to: temporally regulate CRISPR-Cas9 activity post-transfection; reduce off-target effects by disabling the RNP after editing the desired gene; enable spatially confined gene editing via localized delivery of the CRISPR suppressor; reversibly toggle CRISPR activity in live cells and in vivo systems.

The examples provided in the application demonstrate use in editing plasmid and chromosomal DNA in vitro; controlling GFP expression in HEK293 cells; modulating therapeutic editing of VEGFA expression; and utilizing flow cytometry and western blot to validate on/off states.

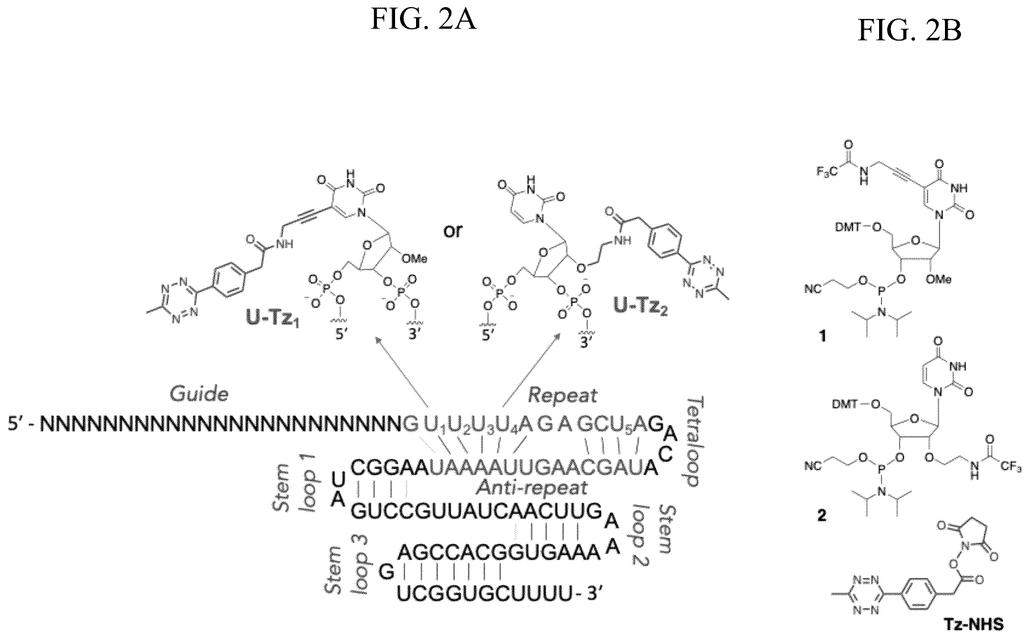

The design of constitutively active Tz-tagged sgRNA was conceived from our exploration of the repeat:anti-repeat region of sgRNA for covalent tagging with Tz (FIG. 2A). Several studies showed that it is a highly sensitive and important area of the sgRNA-Cas9 ribonucleoprotein complex. Nishimasu et al. reported the crystal structure of Cas9 in complex with sgRNA and target DNA. Cas9 consists of two lobes: a recognition (REC) lobe and a nuclease (NUC) lobe. REC1 and REC2 domains of the REC lobe make direct contacts with the repeat:anti-repeat region of sgRNA. Deletion of either the repeat-interacting region or the anti-repeat-interacting region of the REC1 domain abolished the nuclease activity. Sontheimer et al. reported that 2′-OMe RNA modification of all uridines in the repeat:anti-repeat region significantly lowered nuclease activity. Langer and Anderson showed that replacement of the entire repeat:anti-repeat with the corresponding DNA nucleotides completely eliminated nuclease activity. Lastly, Gagnon and Damha reported that replacement of the entire repeat:anti-repeat region with 2′-F-ANA lead to an analogous outcome.

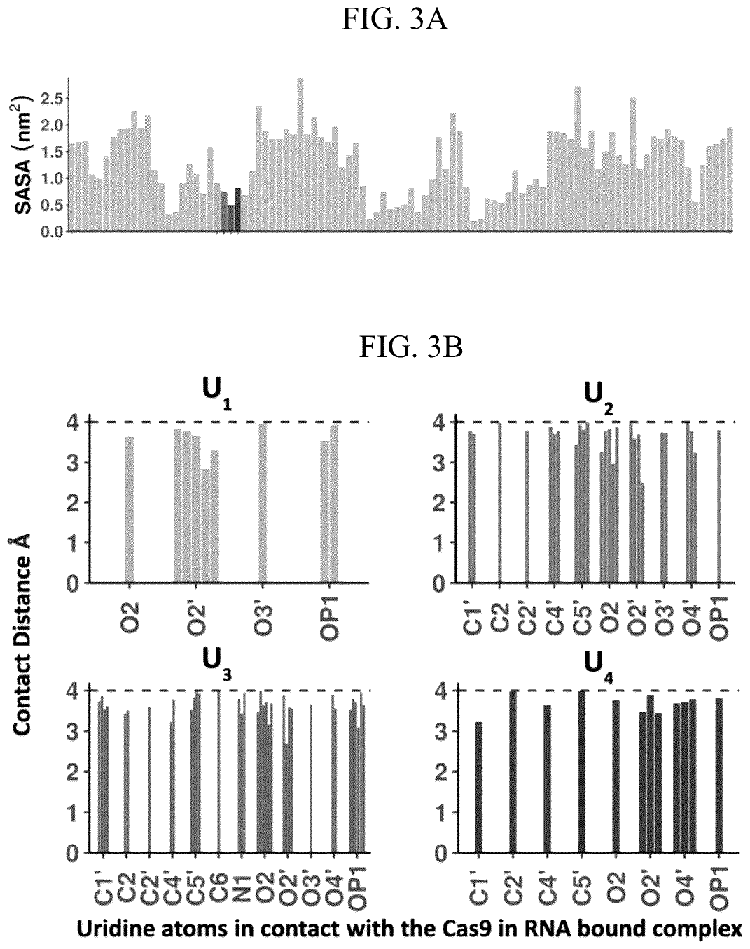

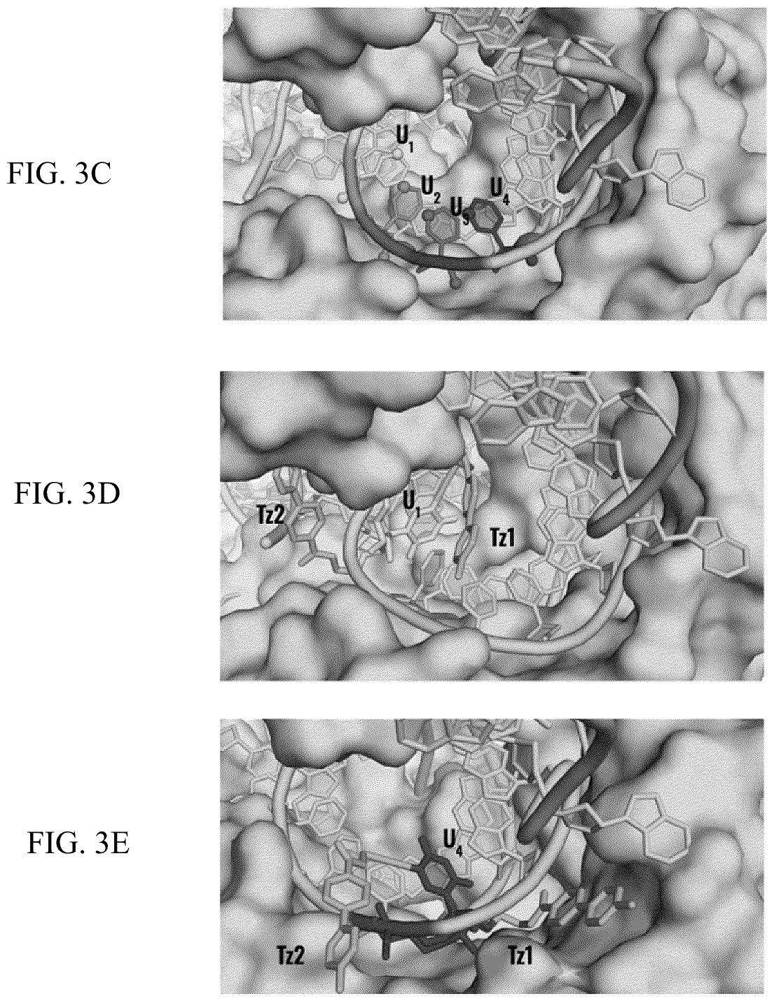

To understand the structural context and evaluate the feasibility of tagging the uridines in the repeat sequence of sgRNA with Tz, we carried out molecular modeling studies utilizing the experimentally solved crystal structure of Cas9:RNA/DNA complex. First, the solvent accessible surface area (SASA) of the uridines in the repeat sequence was calculated. This revealed that U₂ and U₃ have lower SASA compared to U₁ and U₄ (FIG. 3A). U₄ however, is closer to the peripheral surface of the enzyme compared to U₁ (FIG. 3C). Furthermore, the number of contacts with Cas9, defined as any atoms of the Us within 4 Å of the enzyme, is lower for U₁ and U₄ compared to U₂ and U₃ (FIG. 3B). While C5, the site for the Tz₁ modification, has no contacts with the enzyme, the number of contacts for 2′-0, the site for the Tz₂ modification, is higher in case of U₁. Additionally, modeling the Tz₁ and Tz₂ modifications on both U₁ and U₄ revealed that the modifications can be tolerated with minor local rearrangements and minimal disruption of the RNA:enzyme interface in the REC1 domain (FIG. 3D and FIG. 3E). This is particularly true for U₄, where the modified groups point away from the complex. Thus, the modeling predicted that while sgRNA modified with Tz₁ or Tz₂ at U₁ and U₄ should remain active, the modifications might be more accessible for accommodating additional chemistries at position U₄.

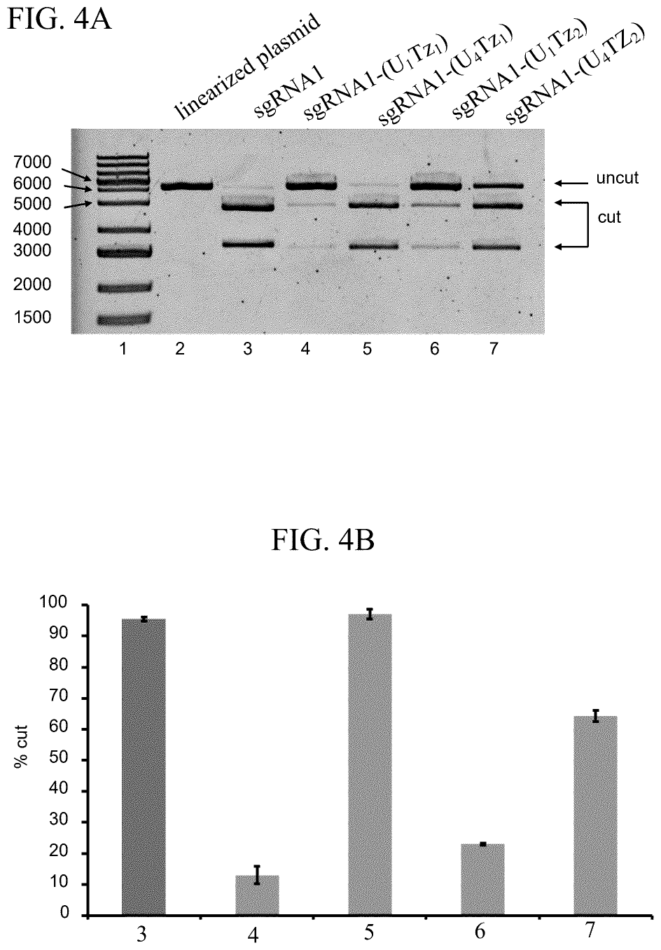

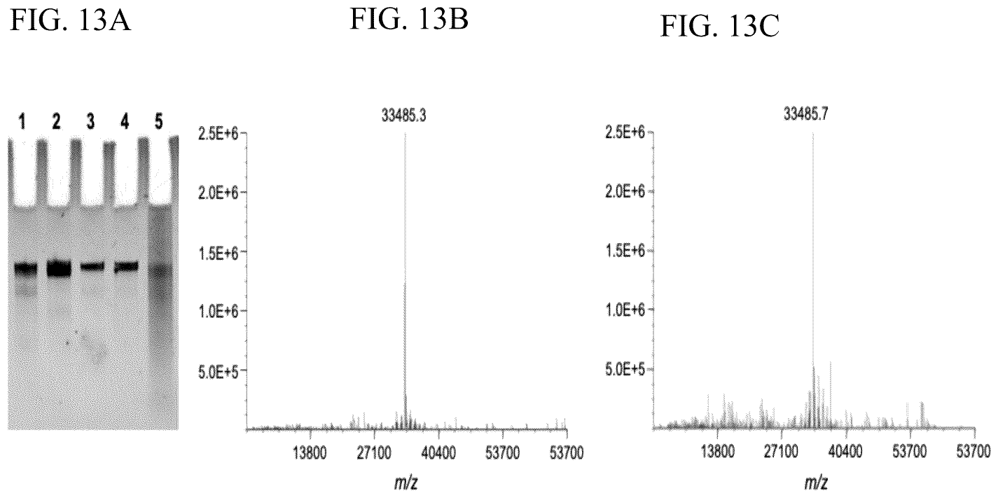

Following these predictions, we synthesized four sgRNAs targeting linearized pBR322 plasmid by solid phase RNA synthesis. The modified uridine phosphoramidites 1 and 2 (FIG. 2B) were incorporated into positions U₁ or U₄. Chemical synthesis of these phosphoramidites has previously been reported. Tz was tagged post-synthetically using Tz-NHS (FIG. 2B). We termed the constructs sgRNA1-(U₁Tz₁), sgRNA1-(U₁Tz₂), sgRNA1-(U₄Tz₁) and sgRNA1-(U₄Tz₂) to indicate the type of Tz modification and the position where it was installed. Following the synthesis, the four Tz-tagged sgRNAs were purified by preparative PAGE. Successful synthesis and purification were confirmed by denaturing gel electrophoresis and ESI-MS (FIG. 13). We subsequently tested their ability to cut linearized pBR322 plasmid in the presence of Cas9. After 16 h of treatment the experiments were analyzed by agarose gel electrophoresis, shown in FIG. 4. sgRNA1-(U₄Tz₁) was found to have the same level of activity as the unmodified sgRNA1. sgRNA1-(U₄Tz₂) was also found to have strong activity that was slightly lower than the unmodified sgRNA1. Meanwhile modification at the U₁-position was either Tz₁ or Tz₂ significantly decreased nuclease activity.

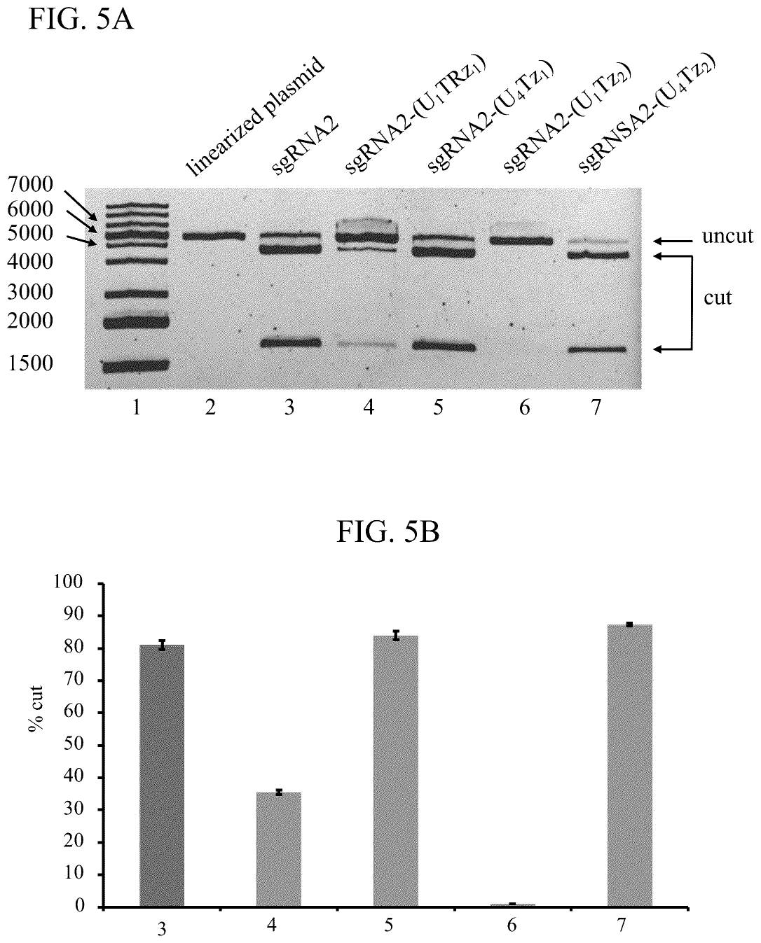

To test the generality of our tagging approach, we synthesized sgRNAs targeting linearized eGFP-N1 plasmid. We analogously termed them sgRNA2-(U₁Tz₁), sgRNA2-(U₁Tz₂), sgRNA2-(U₄Tz₁) and sgRNA2-(U₄Tz₂). Following the synthesis, the four Tz-tagged sgRNAs were purified by preparative PAGE and subsequently tested for their ability to cut linearized eGFP-N1 plasmid in the presence of Cas9. After 16 h of treatment the experiments were analyzed by agarose gel electrophoresis, shown in FIG. 5. Once again, we observed that tagging of U₄ with either Tz₁ or Tz₂ conserved the nuclease activity. Meanwhile, modification at U₁-position with either Tz₁ or Tz₂ significantly lowered nuclease activity. The data indicate a general principle that sgRNAs tagged with either Tz₁ or Tz₂ at the U₄ position of the repeat region will be constitutively active.

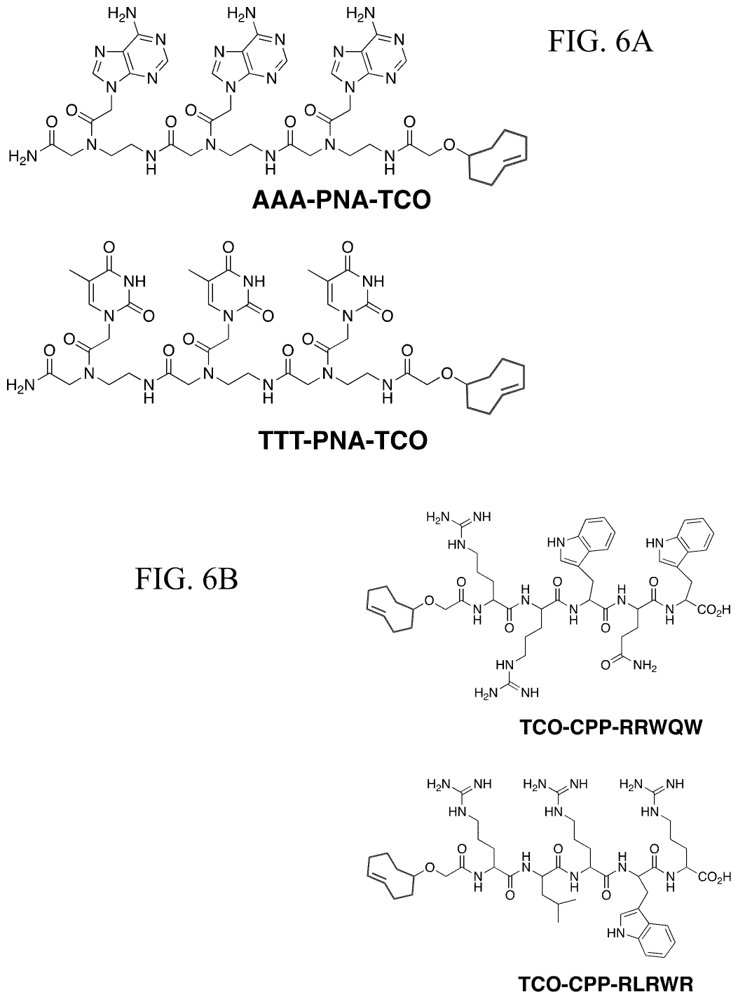

With respect to the design and synthesis of TCO-CRISPR suppressors, after achieving constitutively functional, Tz-tagged sgRNA, we synthesized four TCO-modified CRISPR suppressors, shown in FIG. 6. The compounds have been designed based on 4 principles. They are modular, thus allowing for structural optimization and modification with TCO. They contain functional groups that will interfere with structural elements of sgRNA/Cas9 complex. They are based on the classes of compounds with known cell permeability. They are small molecules with molecular weights under 1200 Da, which is also important to facilitate cell permeability.

The first set of suppressor compounds (FIG. 6A) are peptide nucleic acids (PNAs) containing either thymine or adenine nucleobases. Although long PNA molecules are known to have poor cell permeability, short PNAs have been reported to be cell permeable. We hypothesize that either thymine or adenine nucleobases of the PNAs will interfere with the native repeat-anti-repeat structure that contains five U-A base pairs. Because PNAs can be synthesized in a programmable manner by solid phase synthesis they can be further optimized in terms of length and sequence to find the optimal disrupter of sgRNA/Cas9 complex.

The second set of compounds are TCO-CPP constructs (FIG. 6B) that are based on the smallest-known cell-penetrating peptides (CPPs). They are also small molecules with MW under 1200 Da. As the name implies, they have been shown to be cell permeable and capable of shuttling various therapeutic cargoes across the cellular membrane. These short peptides can be easily synthesized by standard solid phase peptide synthesis. Post-synthetically, they were modified with TCO and purified by HPLC. In addition to inherent steric hinderance, guanidinium groups of the shown TCO-CPP constructs were expected to interfere with nucleobase base-pairing in the repeat:anti-repeat region of sgRNA.

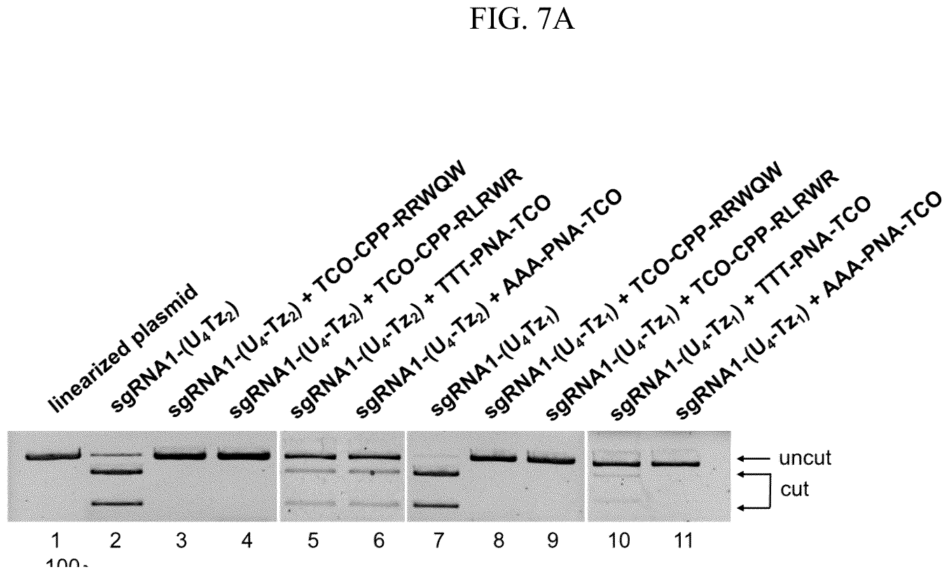

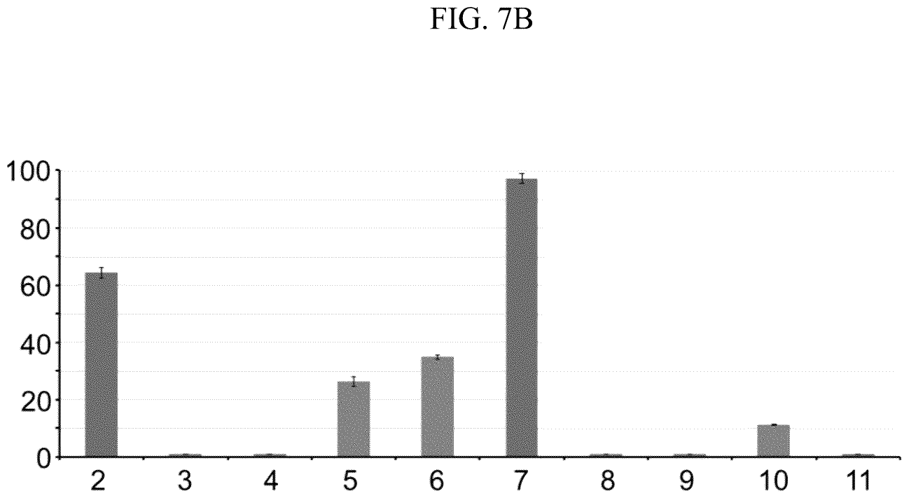

We investigated the impact of TCO-modified CRISPR suppressors on the Cas9-assisted nuclease activity of sgRNA1-(U₄-Tz₁) and sgRNA1-(U₄Tz₂). The pBR322 plasmid was treated with Cas9 and sgRNA1-(U₄-Tz₁) and sgRNA1-(U₄Tz₂) alone or in the presence of TCO-modified CRISPR suppressors. After 16 h of treatment the experiments were analyzed by agarose gel electrophoresis, shown in FIG. 7A. The gel data was quantitated using ImageJ and plotted as a bar graph in FIG. 7B. TCO-CPP-RRWQW (SEQ ID NO: 16) and TCO-CPP-RLRWR (SEQ ID NO: 17) completely inhibited activity of sgRNA1-(U₄Tz₂) (lanes 3 and 4). However, sgRNA1-(U₄Tz₂) treated with TTT-PNA-TCO or AAA-PNA-TCO still retained some residual activity (lanes 5 and 6). In the case of sgRNA1-(U₄Tz₁), addition of TTT-PNA-TCO resulted in a residual nuclease activity (lane 10). TCO-CPP-RRWQW (SEQ ID NO: 16), TCO-CPP-RLRWR (SEQ ID NO: 17) and AAA-PNA-TCO completely inhibited nuclease activity.

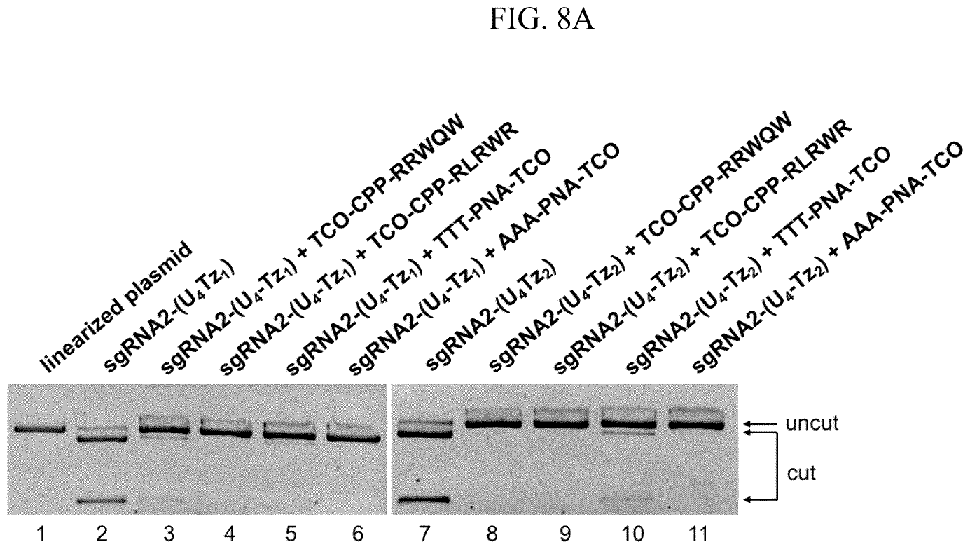

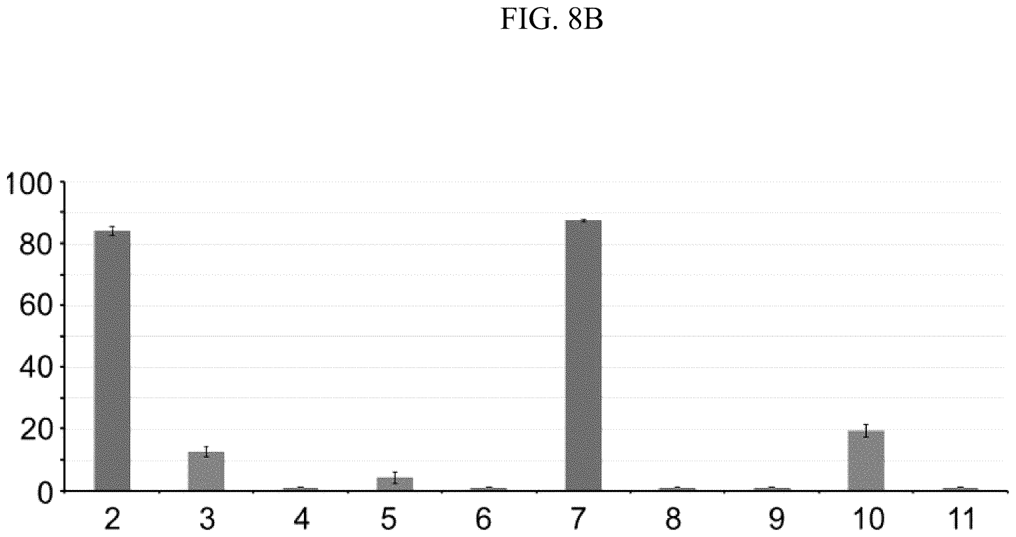

To test the generality of our CRISPR-Cas9 regulation approach, we performed analogous experiments using sgRNA2-(U₄-Tz₁) and sgRNA2-(U₄Tz₂) targeting linearized eGFP-N1 plasmid. After 16 h of treatment the experiments were analyzed by agarose gel electrophoresis, shown in FIG. 8A. The gel data was quantitated using ImageJ and plotted as a bar graph in FIG. 8B. In the case of sgRNA2-(U₄Tz₂), TCO-CPP-RRWQW (SEQ ID NO: 16) and TTT-PNA-TCO addition resulted in a residual nuclease activity (lanes 3 and 5). Addition of TCO-CPP-RLRWR (SEQ ID NO: 17) and AAA-PNA-TCO completely inhibited nuclease activity (lanes 4 and 6). In the case of sgRNA2-(U₄Tz₁), TTT-PNA-TCO partially lowered nuclease activity (lane 10). Meanwhile, TCO-CPP-RRWQW (SEQ ID NO: 16), TCO-CPP-RLRWR (SEQ ID NO: 17) and AAA-PNA-TCO completely inhibited nuclease activity (lanes 8, 9 and 11).

Thus, the in vitro data suggests that temporal control of CRISPR-Cas9 activity, schematically shown in FIG. 1, can be achieved using sgRNA modified with either Tz₁ or Tz₂ at the U₄ position of the repeat region. Inactivation of Tz-tagged sgRNAs can be best achieved using either TCO-CPP-RRWQW (SEQ ID NO: 16) or TCO-CPP-RLRWR (SEQ ID NO: 17).

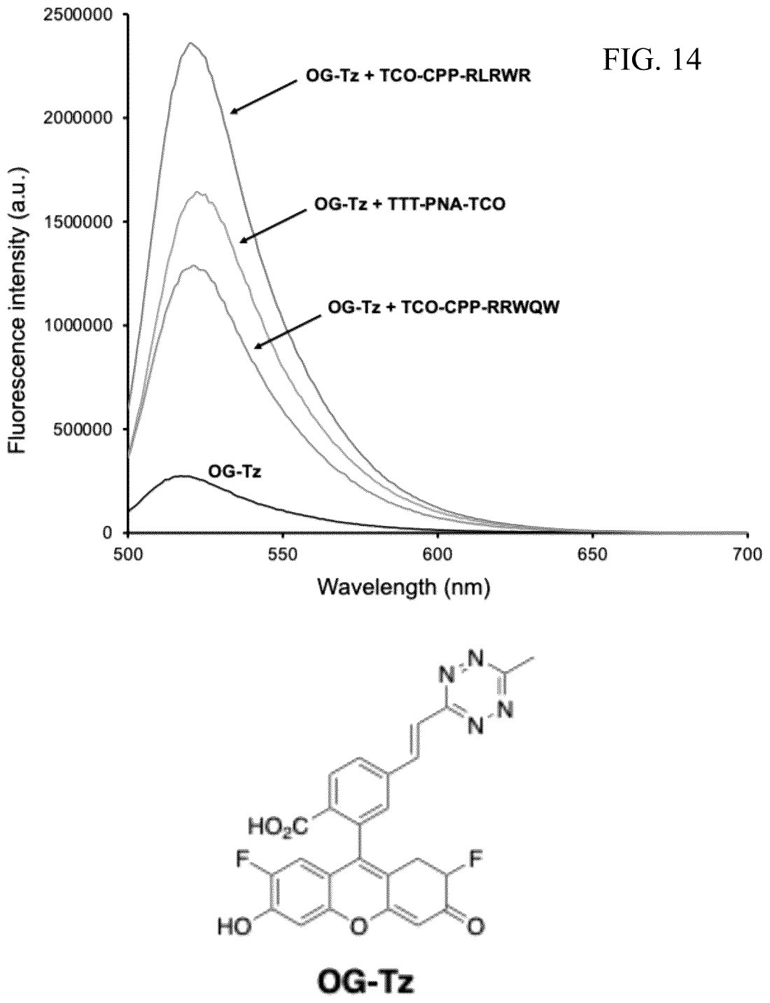

Further, we assessed cell permeability of the TCO-modified CRISPR suppressors using OG-Tz fluorescent probe. It is a reported dye whose fluorescence is quenched by Tz. Fluorescence can be restored upon the click reaction with TCO. We tested fluorescence response of OG-Tz upon addition of the TCO-modified CRISPR suppressors in PBS (pH 7.4). As illustrated in FIG. 14, there is a 10-fold enhancement of fluorescence. The cells were treated with AAA-PNA-TCO, TTT-PNA-TCO, TCO-CPP-RRWQW (SEQ ID NO: 16) or TCO-CPP-RLRWR (SEQ ID NO: 17) for 3 h. Afterwards, the cells were treated with OG-Tz (50 mM) for 2 h. Hoechst 33258 dye was used for nuclear staining. Cellular fluorescence was analyzed using microscopy and flow cytometry.

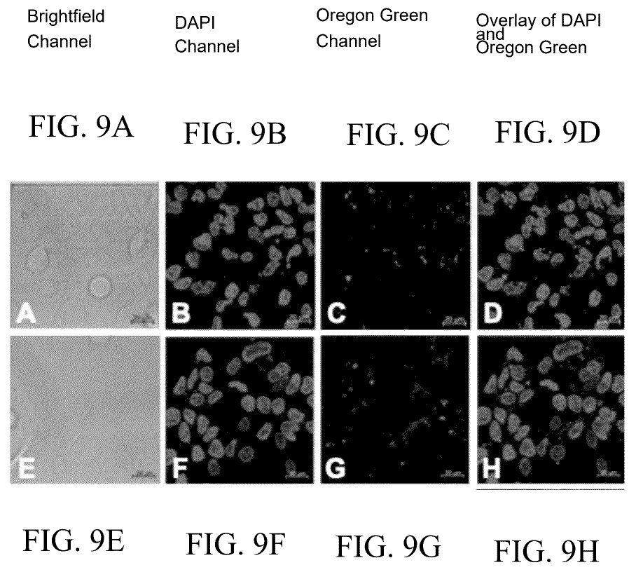

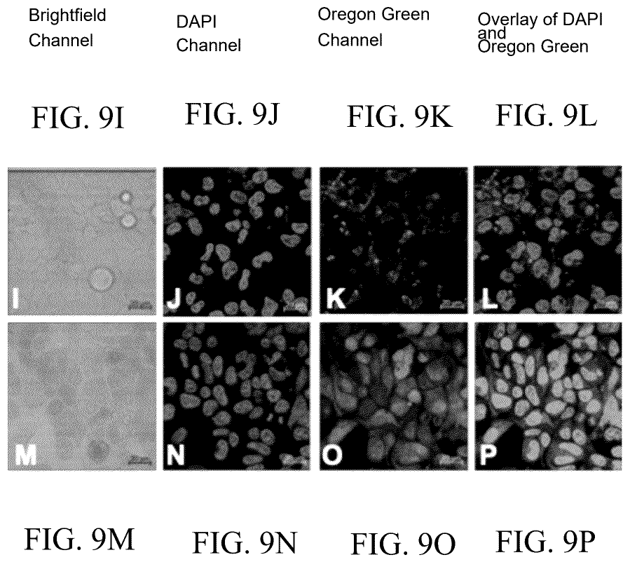

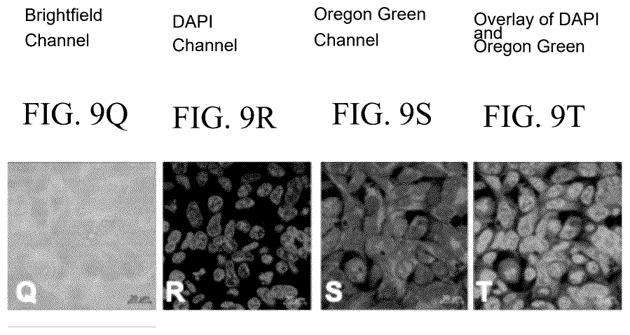

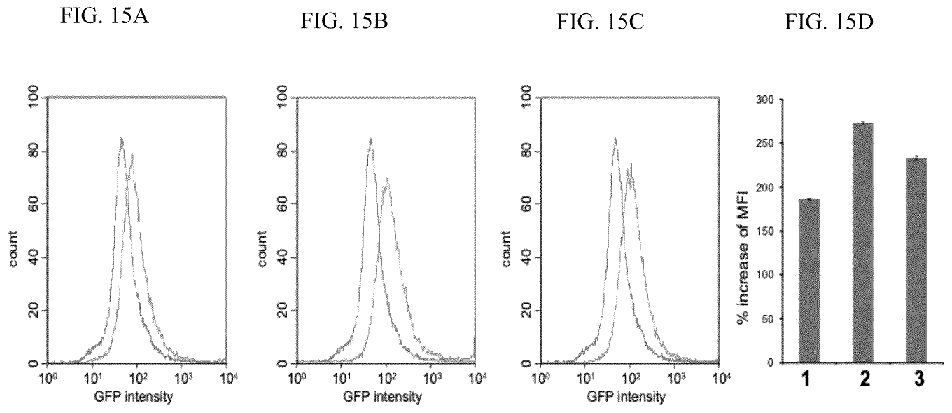

The microscopy data is shown in FIG. 9. As expected, the cells treated with OG-Tz alone showed week fluorescence in the Oregon Green channel (FIG. 9C). Punctate staining was observed in cells treated with Tz-OG and AAA-PNA-TCO and TTT-PNA-TCO, (FIG. 9G and FIG. 9K). The strongest fluorescence was observed in cells treated with OG-Tz and either TCO-CPP-RRWQW (SEQ ID NO: 16) or TCO-CPP-RLRWR (SEQ ID NO: 17) (FIG. 90 and FIG. 9S), suggesting that the CPP compounds have good cell permeability. The cells shown in FIG. 9 were trypsinized and analyzed by flow cytometry, which confirmed the findings (FIG. 15).

Temporal control of CRISPR-Cas9 activity was assessed in GFP-expressing HEK239 cells. We were concerned about post-transfection stability of sgRNA2 that might be exposed to nuclease degradation inside the cells. This concern was addressed by site-specific modification of sgRNA2 with 2′-OMe groups. We followed the strategy described by Yin et al. who identified the exact positions which can be modified with 2′-OMe without significant perturbation to native binding between sgRNA and Cas9. We synthesized sgRNA3, having the sequence: 5′-GGGCGAGGAGCUGUUCACCGGU1U₂U₃U₄AGagcuagaaauagcaaGUUaAaAuAaggcua GUccGUUAucAAcuugaaaaagugGcaccgagucggugcuuuuu-3′ (SEQ ID NO: 15)

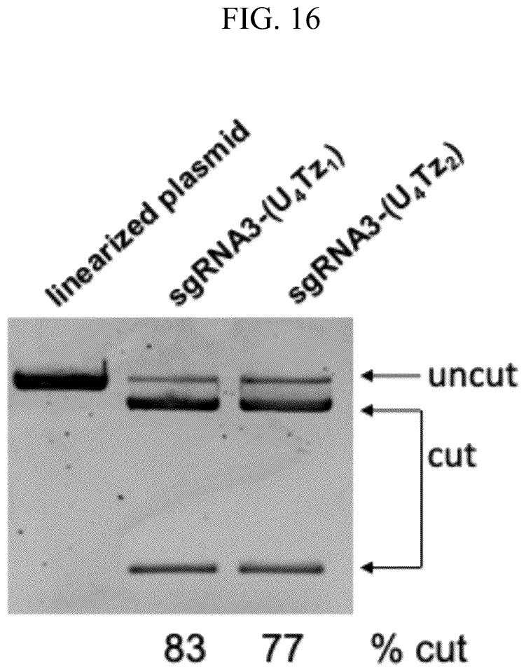

Capital letters indicate unmodified nucleotides, while small letters correspond to nucleotides containing 2′-OMe groups. For reference, uridines of the repeat region are numbered 1-4. U₄ was tagged with either Tz₁ or Tz₂, thus making the constructs sgRNA3-(U₄Tz₁) and sgRNA3-(U₄Tz₂). We assessed the CRISPR-Cas9 activity of these constructs in vitro (FIG. 16). The in vitro experiments showed that 2′-OMe modifications did not perturb nuclease activity and the new constructs behave similarly to sgRNA2-(U₄Tz₁) and sgRNA2-(U₄Tz₂)

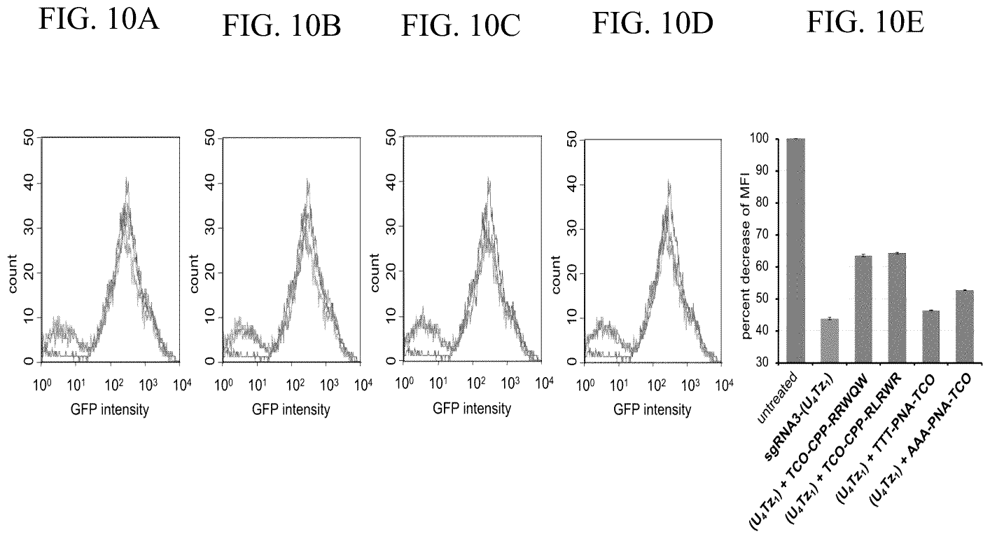

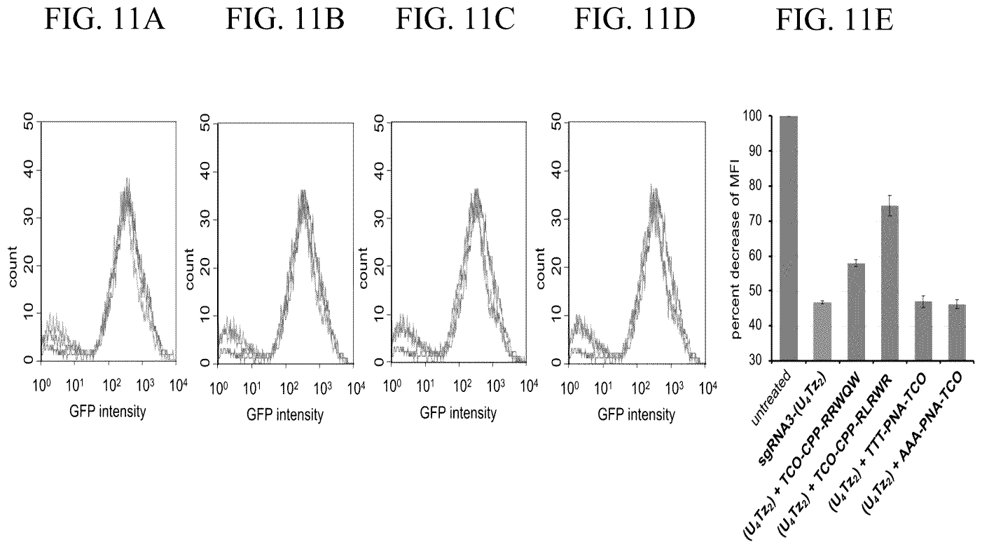

GFP-expressing HEK239 cells were co-transfected with sgRNA3-(U₄Tz₁) or sgRNA3-(U₄Tz₂) and commercially available mRNA that encodes the Cas9 gene for 72 h. After the transfection, the media was replaced with fresh DMEM and the cells were allowed to grow for additional 48 h. GFP expression was analyzed by flow cytometry and compared to the untreated cells. As illustrated in FIG. 9E, the mean fluorescence intensity (MFI) of GFP decreased by 44% after sgRNA3-(U₄Tz₁) treatment. Similarly, MFI of GFP decreased by 47% after sgRNA3-(U₄Tz₂) treatment (FIG. 10E). Ability of TCO-modified CRISPR suppressors to control nuclease activity was examined in the next set of experiments. GFP-expressing HEK239 cells were co-transfected with sgRNA3-(U₄Tz₁) or sgRNA3-(U₄Tz₂) and commercially available mRNA that encodes the Cas9 gene for 48 h. Then, AAA-PNA-TCO, TTT-PNA-TCO, TCO-CPP-RRWQW (SEQ ID NO: 16) and TCO-CPP-RLRWR (SEQ ID NO: 17) (10 mM) were added to the media. After 24 h of treatment with the TCO-modified CRISPR suppressors, the media was replaced with fresh DMEM and the cells were allowed to grow for additional 48 h. A s illustrated in FIG. 10A, FIG. 10E, FIG. 11A and FIG. 11E, TCO-CPP-RRWQW (SEQ ID NO: 16) was able to inactivate sgRNA3-(U₄Tz₁) and sgRNA3-(U₄Tz₂) inside the cells. In these experiments, the MFI of GFP decreased by lower amounts as the result of turned off CRISPR-Cas9 complex. Analogous results were observed after treatment with TCO-CPP-RLRWR (SEQ ID NO: 17) (FIG. 10B, FIG. 10E, FIG. 11B and FIG. 11E). However, the PNA-based CRISPR suppressors were not as effective, as shown in FIG. 10C, FIG. 10D, FIG. 10E, FIG. 11C, FIG. 11D and FIG. 11E. The observed MFI of GFP was very close to the cells treated with sgRNA3-(U₄Tz₁) or sgRNA3-(U₄Tz₂) alone. This is probably due to inferior cell permeability of AAA-PNA-TCO and TTT-PNA-TCO, as was observed in FIG. 9.

### Examples

To illustrate the therapeutic potential of our technology, we implemented it towards a well-established medicinal target, vascular endothelial growth factor A (VEGFA). VEGFA is an angiogenic factor, whose expression is upregulated in neovascular age-related macular degeneration, the leading cause of vision loss. CRISPR-Cas9-based disruption of VEGFA has already been explored as a therapeutic strategy. There are also reported off-target effects associated with a CRISPR-Cas9 system targeting VEGFA. To achieve temporal control over CRISPR-Cas9 treatment, we designed sgRNA4-(U₄Tz₁) and sgRNA4-(U₄Tz₂) where the guide region was programmed to target the VEGFA gene.

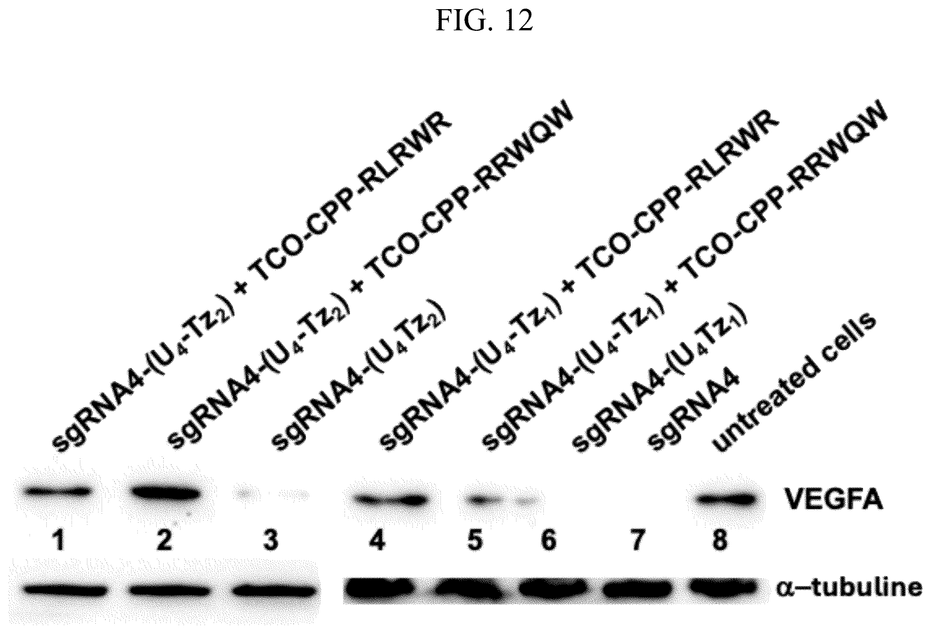

To assess the new constructs, HEK239T cells were co-transfected with the unmodified sgRNA4, sgRNA4-(U₄Tz₁), or sgRNA4-(U₄Tz₂) and commercially available mRNA that encodes the Cas9 gene for 72 h. After the transfection, the media was replaced with fresh DMEM and the cells were allowed to grow for an additional 48 h. VEGFA expression was analyzed by Western blot shown in FIG. 12. We observed marked decrease in VEGFA expression in sgRNA-Cas9 treated cells. The Tz-tagged sgRNAs had similar levels of VEGFA suppression as the unmodified sgRNA4. We repeated these experiments, while adding TCO-CPP-RRWQW (SEQ ID NO: 16) and TCO-CPP-RLRWR (SEQ ID NO: 17) (10 mM) after 48 hours of transfection. VEGFA expression was once again analyzed by Western blot. Addition of TCO-modified CRISPR suppressors resulted in higher VEGFA expression, suggesting that Tz-tagged sgRNAs were inactivated inside the cells.

Herein we described a system that facilitates temporal control of CRISPR-Cas9 nuclease activity using bioorthogonal chemistry. We identified a precise position within the repeat:anti-repeat region of sgRNA that can be tagged with Tz without disrupting Cas9-enabled nuclease activity. We carried out Tz-tagging of three different sgRNAs to illustrate that this strategy is general. Afterwards, we identified TCO-modified small molecule CRISPR suppressors that click to Tz and significantly reduce the nuclease activity. Of the tested molecules, CPP-based suppressors showed the most effective reduction of CRISPR activity both in solution and live cells. Overall, the described strategy to control CRISPR-Cas9 nuclease activity consists of a single small-molecule tag and small molecule cell-permeable suppressor. Lastly, we illustrated the therapeutic potential of our technology by targeting the VEGFA gene in live HEK293T cells. We contemplate that the use of the disclosed technology for temporal control of CRISPR-Cas9 nuclease activity can reduce off-target effects using GUIDE-Seq protocol.

MATERIALS AND METHODS: All oligonucleotide solid phase syntheses were done on a 1.0 μmol scale using the Oligo-800 synthesizer (Azco Biotech, Oceanside, CA, USA). Solid phase syntheses were performed on control-pore glass (CPG-1000) purchased from Glen Research (Sterling, VA, USA). Other oligonucleotide solid phase synthesis reagents were obtained from ChemGenes Corporation (Wilmington, MA, USA). Phosphoramidites (TBDMS as the 2′-OH protecting group): rAwas N-Bz protected, rC was N-Ac protected and rG was N-iBu protected. A, C, G, U phosphoramidites were dissolved in anhydrous acetonitrile (0.07 M) directly before use. m¹A, m⁶A, s²U and s⁴U phosphoramidites were dissolved in anhydrous acetonitrile (0.15 M) directly before use. Coupling step was done using 5-ethylthio-1 H-tetrazole solution (0.25 M) in acetonitrile for 12 min. 5′-detritylation step was done using 3% trichloroacetic acid in CH₂Cl₂. Oxidation step was done using I₂ (0.02 M) in THF/pyridine/H₂O solution.

For gel electrophoresis, 10× Tris/Borate/EDTA (TBE) buffer was purchased from Fisher Scientific Company L.L.C. (Waltham, MA, USA) and used with proper dilution. 30% Arcylamide/Bis-arcylamide solution (29:1) was purchased from Bio-Rad Laboratories, Inc. (Hercules, CA, USA). GeneRuler 1 kb Plus DNA Ladder (cat. #FERSM1331) was purchased from Fisher Scientific. Chromatographic purifications of synthetic materials were conducted using SiliaSphere™ spherical silica gel with an average particle and pore size of 5 μm and 60 Å, respectively (Silicycle Inc, QC, Canada). Thin layer chromatography (TLC) was performed on SiliaPlate™ silica gel TLC plates with 250 μm thickness (Silicycle Inc, QC, Canada). Flash chromatography was performed using Biotage Isolara One instrument (Biotage Sweden AB, Uppsala, Sweden). Preparative TLC was performed using SiliaPlate™ silica gel TLC plates with 1000 μm thickness. ¹H, ¹³C and ³¹P NMR spectroscopy was performed on a Bruker NMR at 500 MHz (¹H) and 126 MHz (¹³C). All ¹³C NMR spectra were proton decoupled. High resolution ESI-MS spectra of small molecules was acquired using Agilent Technologies 6530 Q-TOF instrument.

The peptides, RRWQW (SEQ ID NO: 16) and RLRWR (SEQ ID NO: 17), were purchased from GenScript. PNAs were synthesized using Fmoc-Rink Amide AM Resin (Aapptec, cat. #RRZ001). PNA monomers, Fmoc-PNA-T-OH and Fmoc-PNA-A(Bhoc)-OH), were purchased from Biosearch Technologies (cat. #LK5004-B500 and LK5001-B500). PNAs were synthesized using the procedure described by Braasch, D. A. et al. Current Protocols in Nucleic Acid Chemistry, 2002, 4.11.1-4.11.18.

RNA Sequences:
pBR322-targeting sgRNA:
sgRNA1-(U₁Tz₁)
(SEQ ID NO: 1)
5′-GGGCGCUUGUUUCGGCGUGGGUAGU₁-Tz1U₂U₃U₄AGAGCUA
GACAUAGCAAGUUAAAAUAAGGCUAGUCCGUUAUCAACUUGAAA
AAGUGGCACCGAGUCGGUGCUUUU-3′
sgRNA1-(U₄Tz₁)
(SEQ ID NO: 2)
5′-GGGCGCUUGUUUCGGCGUGGGUAGU₁U₂U3U₄-Tz1AGAGCUA
GACAUAGCAAGUUAAAAUAAGGCUAGUCCGUUAUCAACUUGAAA
AAGUGGCACCGAGUCGGUGCUUUU-3′
sgRNA1-(U₁Tz₂)
(SEQ ID NO: 3)
5′-GGGCGCUUGUUUCGGCGUGGGUAGU₁-Tz2U₂U₃U₄AGAGCUA
GACAUAGCAAGUUAAAAUAAGGCUAGUCCGUUAUCAACUUGAAA
AAGUGGCACCGAGUCGGUGCUUUU-3′
sgRNA1-(U₄Tz₂):
(SEQ ID NO: 4)
5′-GGGCGCUUGUUUCGGCGUGGGUAGU₁U₂U3U₄-Tz2AGAGCUA
GACAUAGCAAGUUAAAAUAAGGCUAGUCCGUUAUCAACUUGAAA
AAGUGGCACCGAGUCGGUGCUUUU-3′
eGFP-targeting sgRNA:
sgRNA2-(U₁Tz₁)
(SEQ ID NO: 5)
5′-GGGCGAGGAGCUGUUCACCGGU₁-Tz1U₂U₃U₄AGAGCUAGAA
AUAGCAAGUUAAAAUAAGGCUAGUCCGUUAUCAACUUGAAAAAG
UGGCACCGAGUCGGUGCUUUUU-3′
sgRNA2-(U₄Tz₁)
(SEQ ID NO: 6)
5′-GGGCGAGGAGCUGUUCACCGGU₁U₂U₃U₄-Tz1AGAGCUAGAA
AUAGCAAGUUAAAAUAAGGCUAGUCCGUUAUCAACUUGAAAAAG
UGGCACCGAGUCGGUGCUUUUU-3′
sgRNA2-(U₁Tz₂)
(SEQ ID NO: 7)
5′-GGGCGAGGAGCUGUUCACCGGU₁-Tz2U₂U₃U₄AGAGCUAGAA
AUAGCAAGUUAAAAUAAGGCUAGUCCGUUAUCAACUUGAAAAAG
UGGCACCGAGUCGGUGCUUUUU-3′
sgRNA2-(U₄Tz₂)
(SEQ ID NO: 8)
5′-GGGCGAGGAGCUGUUCACCGGU₁U₂U₃U₄-Tz2AGAGCUAGAA
AUAGCAAGUUAAAAUAAGGCUAGUCCGUUAUCAACUUGAAAAAG
UGGCACCGAGUCGGUGCUUUUU-3′
eGFP-targeting sgRNA, containing 2′-OMe
groups (small letters):_sgRNA3
(SEQ ID NO: 9)
5′-GGGCGAGGAGCUGUUCACCGGUUUUAGagcuagaaauagca
aGUUaAaAuAaggcuaGUccGUUAucAAcuugaaaaagugGcac
cgagucggugcuuuuu-3′
sgRNA3-(U₄Tz₁)
(SEQ ID NO: 10)
5′-GGGCGAGGAGCUGUUCACCGGUUUU₄-Tz1AGagcuagaaa
uagcaaGUUaAaAuAaggcuaGUccGUUAucAAcuugaaaaag
ugGcaccgagucggugcuuuuu-3′
sgRNA3-(U₄Tz₂)
(SEQ ID NO: 11)
5′-GGGCGAGGAGCUGUUCACCGGUUUU₄-Tz2AGagcuagaaa
uagcaaGUUaAaAuAaggcuaGUccGUUAucAAcuugaaaaag
ugGcaccgagucggugcuuuuu-3′
VEGFA-targeting sgRNA, containing 2′-OMe
groups (small letters):_sgRNA4
(SEQ ID NO: 12)
5′-GGUGAGUGAGUGUGUGCGUGGUUUUAGagcuagaaauagca
aGUUaAaAuAaggcuaGUccGUUAucAAcuugaaaaagugGcac
cgagucggugcuuuuu-3′
sgRNA4-(U₄Tz₁)
(SEQ ID NO: 13)
5′-GGUGAGUGAGUGUGUGCGUGGUUUU₄-Tz1AGagcuagaaa
uagcaaGUUaAaAuAaggcuaGUccGUUAucAAcuugaaaaag
ugGcaccgagucggugcuuuuu-3′
sgRNA4-(U₄Tz₂)
(SEQ ID NO: 14)
5′-GGUGAGUGAGUGUGUGCGUGGUUUU₄-Tz2AGagcuagaaa
uagcaaGUUaAaAuAaggcuaGUccGUUAucAAcuugaaaaag
ugGcaccgagucggugcuuuuu-3′

Capital letters indicate unmodified nucleotides, while small letters correspond to nucleotides containing 2′-OMe groups.

CRISPR-Cas9 in vitro DNA cleavage assay: eGFP-N1 plasmid DNA (10 U/μL, 1 μL, NEB, R3510L) was diluted with water (16.87 μL) and NEB buffer 3.1 (10×, 2 μL). The plasmid was linearized directly prior to CRISPR with Dralll-HF (10 U/μL, 1 μL, NEB, R3510L). For the Cas9-mediated DNA cleavage assay, sgRNA (300 nM, 5 μL), Cas9 (1 μM, 0.3 μL, NEB, M0386S), Cas9 buffer (10×, 1 μL, NEB), linearized plasmid (20 nM, 1.5 μL) and MQ H₂O (2.2 μL) were mixed (final volume=10 μL) and incubated for 16 h at 37° C. CRISPR experiments were terminated by the addition of proteinase K (20 mg/mL, 0.5 μL) for 1 h at 37° C. The reaction (10 μL) was mixed with blue loading buffer (6×, 2 μL, NEB, B7703S) and loaded on a 1% agarose stained with ethidium bromide (1×TBE running buffer).

CRISPR-Cas9 experiments in HEK293 cells: CRISPR-Cas9 experiments, were carried out following the procedure reported by Yin, H. et al. [Nat. Chem. Biol. 2018, 14, 311-316]. The GFP-expressing HEK293 cells were purchased from GenTarget (cat #SC001) and cultured in DMEM, containing 10% FBS and 1× Penicillin/Streptomycin, at 37° C., 5% CO₂, and 95% humidity. The cells were seeded at a concentration of 1×10⁵ cells per well in 6-well plate 24 h prior to the experiment. The cells were transfected with Cas9 mRNA (500 ng, Thermo Fisher Scientific), GFP-targeting sgRNAs (30 nM) using lipofectamine (1.5 μL) (Invitrogen™ LMRNA003) for 72 h in Opti-MEM reduced serum media. After 72 h, Opti-MEM was replaced with fresh DMEM and the cells were grown for additional 48 h. The cells were treated with trypsin for 5 min, collected by centrifugation at 1000 RPM and suspended in PBS (1 mL). GFP expression was analyzed by flow cytometry. Data from 10⁶ cells were acquired using a FACS Aria III cell sorter equipped with a 488 nm/blue coherent sapphire solid-state laser, 20 mW (BD Biosciences, San Jose, CA, USA). Data analyses were carried out using FlowJo software (Ashland, OR, USA), according to manufacturer's instructions. Parameters, such as MFI and the percentages of specific populations were quantified by histogram analysis.

Ability of TCO-modified CRISPR suppressors to control nuclease activity was examined as follows: GFP-expressing HEK239 cells were co-transfected with Cas9 mRNA (500 ng, Thermo Fisher Scientific), GFP-targeting sgRNAs (30 nM) using lipofectamine (1.5 μL) (Invitrogen™ LMRNA003) for 48 h. Then, AAA-PNA-TCO, TTT-PNA-TCO, TCO-CPP-RRWQW (SEQ ID NO: 16) and TCO-CPP-RLRWR (SEQ ID NO: 17) (10 mM) were added to the media. After 24 h of treatment with the TCO-modified CRISPR suppressors, the media was replaced with fresh DMEM and the cells were allowed to grow for additional 48 h. The cells were treated with trypsin for 5 min, collected by centrifugation at 1000 RPM and suspended in PBS (1 mL). GFP expression was analyzed by flow cytometry. Data from 10⁶ cells were acquired using a FACS Aria III cell sorter equipped with a 488 nm/blue coherent sapphire solid-state laser, 20 mW (BD Biosciences, San Jose, CA, USA). Data analyses were carried out using FlowJo software (Ashland, OR, USA), according to manufacturer's instructions. Parameters, such as MFI and the percentages of specific populations were quantified by histogram analysis.

Assessment of cell permeability of TCO-modified CRISPR suppressors: The HEK293 cells were cultured in DMEM, containing 10% FBS and 1× Penicillin/Streptomycin, at 37° C., 5% CO₂, and 95% humidity. The cells were seeded at a concentration of 1×10⁵ cells per well in 6-well plate 24 h prior to the experiment. The cells were treated with the TCO-modified CRISPR suppressors (30 mM) for 3 h. Then, the media was replaced and the cells were treated with OG-Tz (50 mM) for 2h. Afterwards, the cells were treated with trypsin for 5 min, collected by centrifugation at 1000 RPM and suspended in PBS (1 mL). GFP expression was analyzed by flow cytometry. Data from 10⁶ cells were acquired using a FACS Aria III cell sorter equipped with a 488 nm/blue coherent sapphire solid-state laser, 20 mW (BD Biosciences, San Jose, CA, USA). Data analyses were carried out using FlowJo software (Ashland, OR, USA), according to manufacturer's instructions. Parameters, such as MFI and the percentages of specific populations were quantified by histogram analysis.

HEK293 cells were grown to ˜70% confluence in 35 mm MatTek glass bottom dishes in DMEM supplemented with 10% bovine serum albumin, penicillin and streptomycin. The cells were treated with AAA-PNA-TCO, TTT-PNA-TCO, TCO-CPP-RRWQW (SEQ ID NO: 16) or TCO-CPP-RLRWR (SEQ ID NO: 17) (30 mM) for 3 h. Afterwards, the cells were treated with OG-Tz (50 mM) for 2 h. Hoechst 33258 dye was used for nuclear staining. Cellular fluorescence was analyzed using confocal microscopy. Microscopy experiments were carried out using Zeiss LSM980 confocal microscope.

Modeling Studies: The structure of the Cas9 in complex with RNA and DNA was obtained from the Protein Data Bank (PDB ID: 4008; Nishimasu, H. et al. 2014, Cell, 156(5), 935-949). The solvent accessible surface area (SASA) was computed using the sasa module from GROMACS (Lindahl, Abraham, Hess & van der Spoel Version 2020, doi: 10.5281/zenodo.3562512) employing the default probe radius of 0.14 nm. This radius is representative of water which is the primary solvent in the system under study. The number and distance of atomic contacts were calculated for the RNA bound to Cas9 using PyMOL (The PyMOL Molecular Graphics System, Version 3.0 Schrödinger, LLC). A cutoff distance of 4 Å was used to identify RNA:protein contacts. The Tz1 and Tz2 modifications were introduced on the RNA repeat sequence in MOE (Molecular Operating Environment (MOE), 2024.06 Chemical Computing Group ULC, 910-1010 Sherbrooke St. W, Montreal, QC H3A2R7, 2024). Local minimization of the structure was then carried out to accommodate the modifications in the complex.

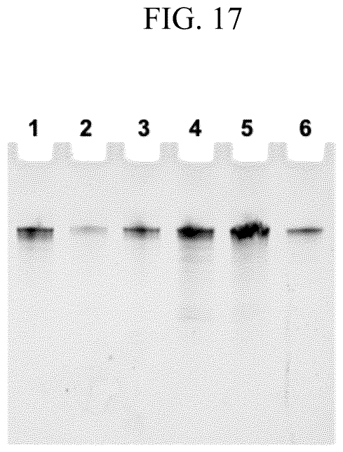

Modification of sgRNAs with Tz: sgRNA (263 μM) was dissolved in borate buffer (250 μL, pH=9.4). Compound Tz-NHS was dissolved in DMF (400 μL, 50 mM) and added to the RNA. The reaction mixture was vortexed and agitated at 1000 rpm at rt for 8 h. The conjugated sgRNA was purified from small molecules using Amicon Ultra 3K column (MilliporeSigma, cat #UFC500396) and washed with MQ H₂O (3× 300 μL). sgRNAs were purified by preparative PAGE. Purified sgRNAs were analyzed using analytical PAGE, as shown in FIG. 13 and FIG. 17.

Western blot analysis: Total protein lysate was harvested in RIPA buffer and proteins separated by 10% SDS-PAGE were transferred to a PVDF membrane by using High MW protocol on Biorad Turbo wet transfer. Following transfer, membranes were blocked in 5% milk in PBST for 1 h at room temperature, washed three times in PBS with 0.1% (w/v) Tween 20 (PBS/T) for 10 min each and placed in primary antibody overnight at 4° C. Primary antibody was rabbit VEGFA polyclonal antibody (1:2000 dilution, Cat no:19003-1-AP, Proteintech) and a-tubulin monoclonal antibody (1:20,000 dilution, Cat no. 666031-1-Ig, Proteintech) in 5% BSA in PBS/T. Following overnight incubation, membranes were washed three times in PBS/T for 10 min each. Then membranes were incubated in a 1:10,000 dilution of goat anti-rabbit secondary (Jackson), goat-anti mouse secondary in 5% milk and PBS/T. Membranes were washed a final 3 times in PBS/T for 10 min each prior to being imaged on the BioRad ChemiDoc by using Thermo Scientific SuperSignal West Pico PLUS Chemiluminescent Substrate (Catalog: 34577).

### REFERENCES

Cong, L.; Ran, F. A.; Cox, D.; Lin, S.; Barretto, R.; Habib, N.; Hsu, P. D.; Wu, X.; Jiang, W.; Marraffini, L. A.; Zhang, F. Multiplex Genome Engineering Using CRISPR/Cas Systems. Science, 2013, 339 (6121), 819-823. Mali, P.; Yang, L.; Esvelt, K. M.; Aach, J.; Guell, M.; DiCarlo, J. E.; Norville, J. E.; Church, G. M. RNA-Guided Human Genome Engineering via Cas9. Science, 2013, 339 (6121), 823-826. Lu, Y; Xue, J.; Deng, T.; Zhou, X.; Yu, K.; Deng, L.; Huang, M.; Yi, X.; Liang, M.; Wang, Y; Shen, H.; Tong, R.; Wang, W.; Li, L.; Song, J.; Li, J.; Su, X.; Ding, Z.; Gong, Y; Zhu, J.; Wang, Y; Zou, B.; Zhang, Y; Li, Y; Zhou, L.; Liu, Y; Yu, M.; Wang, Y; Zhang, X.; Yin, L.; Xia, X.; Zeng, Y; Zhou, Q.; Ying, B.; Chen, C.; Wei, Y; Li, W; Mok, T. Safety and Feasibility of CRISPR-Edited T Cells in Patients with Refractory Non-Small-Cell Lung Cancer. Nat. Med. 2020, 26 (5), 732-740. Lacey, S. F.; Fraietta, J. A. First Trial of CRISPR-Edited T Cells in Lung Cancer. Trends Mol. Med. 2020, 713-715. Stadtmauer, E. A.; Fraietta, J. A.; Davis, M. M.; Cohen, A. D.; Weber, K. L.; Lancaster, E.; Mangan, P. A.; Kulikovskaya, I.; Gupta, M.; Chen, F.; Tian, L.; Gonzalez, V. E.; Xu, J.; Jung, I. young; Joseph Melenhorst, J.; Plesa, G.; Shea, J.; Matlawski, T.; Cervini, A.; Gaymon, A. L.; Desjardins, S.; Lamontagne, A.; Salas-Mckee, J.; Fesnak, A.; Siegel, D. L.; Levine, B. L.; Jadlowsky, J. K.; Young, R. M.; Chew, A.; Hwang, W. T; Hexner, E. O.; Carreno, B. M.; Nobles, C. L.; Bushman, F. D.; Parker, K. R.; Qi, Y; Satpathy, A. T.; Chang, H. Y; Zhao, Y; Lacey, S. F.; June, C. H. CRISPR-Engineered T Cells in Patients with Refractory Cancer. Science, 2020, 367 (6481). Demirci, S.; Leonard, A.; Haro-Mora, J. J.; Uchida, N.; Tisdale, J. F. CRISPR/Cas9 for Sickle Cell Disease: Applications, Future Possibilities, and Challenges. In Adv. Exp. Med. Biol. 2019, 1144, 37-52. Frangoul, H.; Altshuler, D.; Cappellini, M. D.; Chen, Y-S.; Domm, J.; Eustace, B. K.; Foell, J.; de la Fuente, J.; Grupp, S.; Handgretinger, R.; Ho, T. W; Kattamis, A.; Kernytsky, A.; Lekstrom-Himes, J.; Li, A. M.; Locatelli, F.; Mapara, M. Y; de Montalembert, M.; Rondelli, D.; Sharma, A.; Sheth, S.; Soni, S.; Steinberg, M. H.; Wall, D.; Yen, A.; Corbacioglu, S. CRISPR-Cas9 Gene Editing for Sickle Cell Disease and β-Thalassemia. New England J. Med. 2021, 384 (3), 252-260. Tsai, S. Q.; Zheng, Z.; Nguyen, N. T.; Liebers, M.; Topkar, V. V; Thapar, V.; Wyvekens, N.; Khayter, C.; lafrate, A. J.; Le, L. P.; Aryee, M. J.; Joung, J. K. GUIDE-Seq Enables Genome-Wide Profiling of off-Target Cleavage by CRISPR-Cas Nucleases. Nat. Biotechnol. 2015, 33 (2), 187-197. Klein, M.; Eslami-Mossallam, B.; Arroyo, D. G.; Depken, M. Hybridization Kinetics Explains CRISPR-Cas Off-Targeting Rules. Cell Rep. 2018, 22 (6), 1413-1423. Boyle, E. A.; Andreasson, J. O. L.; Chircus, L. M.; Sternberg, S. H.; Wu, M. J.; Guegler, C. K.; Doudna, J. A.; Greenleaf, W. J. High-Throughput Biochemical Profiling Reveals Sequence Determinants of DCas9 off-Target Binding and Unbinding. Proc. Nat. Acad. Sci. 2017, 114 (21), 5461-5466. Maji, B.; Moore, C. L.; Zetsche, B.; Volz, S. E.; Zhang, F.; Shoulders, M. D.; Choudhary, A. Multidimensional Chemical Control of CRISPR-Cas9. Nat. Chem. Biol. 2017, 13 (1), 9-11. Mendoza, S. D.; Nieweglowska, E. S.; Govindarajan, S.; Leon, L. M.; Berry, J. D.; Tiwari, A.; Chaikeeratisak, V.; Pogliano, J.; Agard, D. A.; Bondy-Denomy, J. A Bacteriophage Nucleus-like Compartment Shields DNA from CRISPR Nucleases. Nature, 2020, 577 (7789), 244-248. Nakamura, M.; Srinivasan, P.; Chavez, M.; Carter, M. A.; Dominguez, A. A.; La Russa, M.; Lau, M. B.; Abbott, T. R.; Xu, X.; Zhao, D.; Gao, Y; Kipniss, N. H.; Smolke, C. D.; Bondy-Denomy, J.; Qi, L. S. Anti-CRISPR-Mediated Control of Gene Editing and Synthetic Circuits in Eukaryotic Cells. Nat. Commun. 2019, 10 (1). Wu, Y; Yang, L.; Chang, T.; Kandeel, F.; Yee, J. K. A Small Molecule-Controlled Cas9 Repressible System. Mol. Ther. Nucleic Acids, 2020, 19, 922-932. Iwasaki, R. S.; Ozdilek, B. A.; Garst, A. D.; Choudhury, A.; Batey, R. T. Small Molecule Regulated SgRNAs Enable Control of Genome Editing in E. Coli by Cas9. Nat. Commun. 2020, 11 (1), 1394. Kundert, K.; Lucas, J. E.; Watters, K. E.; Fellmann, C.; Ng, A. H.; Heineike, B. M.; Fitzsimmons, C. M.; Oakes, B. L.; Qu, J.; Prasad, N.; Rosenberg, O. S.; Savage, D. F.; EI-Samad, H.; Doudna, J. A.; Kortemme, T. Controlling CRISPR-Cas9 with Ligand-Activated and Ligand-Deactivated SgRNAs. Nat. Commun. 2019, 10 (1). Liu, X.; Xiong, W.; Qi, Q.; Zhang, Y; Ji, H.; Cui, S.; An, J.; Sun, X.; Yin, H.; Tian, T.; Zhou, X. Rational Guide RNA Engineering for Small-Molecule Control of CRISPR/Cas9 and Gene Editing. Nucleic Acids Res. 2022, 50 (8), 4769-4783. Czuban, M.; Srinivasan, S.; Yee, N. A.; Agustin, E.; Koliszak, A.; Miller, E.; Khan, I.; Quinones, I.; Noory, H.; Motola, C.; Volkmer, R.; Di Luca, M.; Trampuz, A.; Royzen, M.; Mejia Oneto, J. M. Bio-Orthogonal Chemistry and Reloadable Biomaterial Enable Local Activation of Antibiotic Prodrugs and Enhance Treatments against Staphylococcus Aureus Infections. ACS Cent. Sci. 2018, 4 (12), 1624-1632. Oneto, J. M. M.; Khan, I.; Seebald, L.; Royzen, M. In Vivo Bioorthogonal Chemistry Enables Local Hydrogel and Systemic Pro-Drug to Treat Soft Tissue Sarcoma. ACS Cent. Sci. 2016, 2 (7), 476-482. Nishimasu, H.; Ran, F. A.; Hsu, P. D.; Konermann, S.; Shehata, S. I.; Dohmae, N.; Ishitani, R.; Zhang, F.; Nureki, O. Crystal Structure of Cas9 in Complex with Guide RNA and Target DNA. Cell, 2014, 156 (5), 935-949. Mir, A.; Alterman, J. F.; Hassler, M. R.; Debacker, A. J.; Hudgens, E.; Echeverria, D.; Brodsky, M. H.; Khvorova, A.; Watts, J. K.; Sontheimer, E. J. Heavily and Fully Modified RNAs Guide Efficient SpyCas9-Mediated Genome Editing. Nat. Commun. 2018, 9 (1). Yin, H.; Song, C. Q.; Suresh, S.; Kwan, S. Y; Wu, Q.; Walsh, S.; Ding, J.; Bogorad, R. L.; Zhu, L. J.; Wolfe, S. A.; Koteliansky, V.; Xue, W.; Langer, R.; Anderson, D. G. Partial DNA-Guided Cas9 Enables Genome Editing with Reduced off-Target Activity. Nat. Chem. Biol. 2018, 14 (3), 311-316. O'Reilly, D.; Kartje, Z. J.; Ageely, E. A.; Malek-Adamian, E.; Habibian, M.; Schofield, A.; Barkau, C. L.; Rohilla, K. J.; Derossett, L. B.; Weigle, A. T.; Damha, M. J.; Gagnon, K. T. Extensive CRISPR RNA Modification Reveals Chemical Compatibility and Structure-Activity Relationships for Cas9 Biochemical Activity. Nucleic Acids Res. 2019, 47 (2), 546-558. Hoy, A.; Zheng, Y Y; Sheng, J.; Royzen, M. Bio-Orthogonal Chemistry Conjugation Strategy Facilitates Investigation of N-Methyladenosine and Thiouridine Guide RNA Modifications on CRISPR Activity. CRISPR J. 2022, 5 (6), 787-798. Good, L.; Nielsen, P. E. Inhibition of Translation and Bacterial Growth by Peptide Nucleic Acid Targeted to Ribosomal RNA. Proc. Nat. Acad. Sci. 1998, 95 (5), 2073-2076. Liu, B. R.; Huang, Y W.; Aronstam, R. S.; Lee, H. J. Identification of a Short Cell-Penetrating Peptide from Bovine Lactoferricin for Intracellular Delivery of DNA in Human A549 Cells. PLoS One, 2016, 11 (3). Wang, Y F.; Xu, X.; Fan, X.; Zhang, C.; Wei, Q.; Xi, W.; Guo, W.; Wei, X.; Yu, J.; Yan, J. L.; Liang, H. P. A Cell-Penetrating Peptide Suppresses Inflammation by Inhibiting NF-KB Signaling. Mol. Ther. 2011, 19 (10), 1849-1857. Fang, B.; Guo, H. Y; Zhang, M.; Jiang, L.; Ren, F. Z. The Six Amino Acid Antimicrobial Peptide BLFcin6 Penetrates Cells and Delivers SiRNA. FEBS J. 2013, 280 (4), 1007-1017. Wu, H.; Yang, J.; Šečkute, J.; Devaraj, N. K. In Situ Synthesis of Alkenyl Tetrazines for Highly Fluorogenic Bioorthogonal Live-Cell Imaging Probes. Angew. Chem. Int. Ed. 2014, 53 (23), 5805-5809. Yin, H.; Song, C.-Q.; Suresh, S.; Wu, Q.; Walsh, S.; Rhym, L. H.; Mintzer, E.; Bolukbasi, M. F.; Zhu, L. J.; Kauffman, K.; Mou, H.; Oberholzer, A.; Ding, J.; Kwan, S.-Y; Bogorad, R. L.; Zatsepin, T.; Koteliansky, V.; Wolfe, S. A.; Xue, W; Langer, R.; Anderson, D. G. Structure-Guided Chemical Modification of Guide RNA Enables Potent Non-Viral in Vivo Genome Editing. Nat. Biotechnol. 2017, 35 (12), 1179-1187. Miller, J. W.; Le Couter, J.; Strauss, E. C.; Ferrara, N. Vascular Endothelial Growth Factor A in Intraocular Vascular Disease. Ophthalmology 2013, 120 (1), 106-114. org 10.1016 j.ophtha.2012.07. Bhutto, I. A.; McLeod, D. S.; Hasegawa, T.; Kim, S. Y; Merges, C.; Tong, P.; Lutty, G. A. Pigment Epithelium-Derived Factor (PEDF) and Vascular Endothelial Growth Factor (VEGF) in Aged Human Choroid and Eyes with Age-Related Macular Degeneration. Exp. Eye Res. 2006, 82 (1), 99-110. Zeng, Z.; Li, S.; Ye, X.; Wang, Y; Wang, Q.; Chen, Z.; Wang, Z.; Zhang, J.; Wang, Q.; Chen, L.; Zhang, S.; Zou, Z.; Lin, M.; Chen, X.; Zhao, G.; McAlinden, C.; Lei, H.; Zhou, X.; Huang, J. Genome Editing VEGFA Prevents Corneal Neovascularization In Vivo. Adv. Sci. 2024, 11 (25). Chung, S. H.; Sin, T.-N.; Dang, B.; Ngo, T.; Lo, T.; Lent-Schochet, D.; Meleppat, R. K.; Zawadzki, R. J.; Yiu, G. CRISPR-Based VEGF Suppression Using Paired Guide RNAs for Treatment of Choroidal Neovascularization. Mol. Ther. Nucleic Acids, 2022, 28, 613-622.

What is claimed is:

1. A method for temporally controlling CRISPR-Cas9 nuclease activity in a cell, comprising: providing a first composition comprising a single guide RNA (sgRNA) covalently tagged with a first bioorthogonal reactive group at a nucleotide within the repeat region of the sgRNA;
providing a second composition comprising a small molecule CRISPR suppressor comprising a second bioorthogonal reactive group;
forming a ribonucleoprotein (RNP) complex by complexing the sgRNA with a Cas9 protein;
introducing the RNP complex into a cell; subsequently administering the second composition to the cell; wherein the first and second reactive groups are members of a bioorthogonal click chemistry pair that react under physiological conditions to form a covalent adduct, and wherein formation of the adduct inhibits CRISPR-Cas9 nuclease activity.

2. A kit-of-parts composition comprising: a first component including a Cas9 protein and a single guide RNA (sgRNA) comprising a first bioorthogonal reactive group covalently attached to a nucleotide within the repeat region of the sgRNA; a second component including a small molecule CRISPR suppressor comprising a second bioorthogonal reactive group; wherein the first and second reactive groups are capable of undergoing a bioorthogonal click reaction under physiological conditions to inhibit CRISPR-Cas9 nuclease activity.

3. A composition comprising: a Cas9 protein; a single guide RNA (sgRNA) covalently linked to a small molecule CRISPR suppressor through a bioorthogonal click chemistry reaction product; wherein the covalent adduct is formed by reaction of a first reactive group covalently attached to a nucleotide within the sgRNA and a second reactive group on the small molecule suppressor, and wherein the adduct inhibits CRISPR Cas9 nuclease activity.

4. The method of claim 1, wherein the reactive pair is a cyclooctyne and an azide.

5. The method of claim 1, wherein the first reactive group is a tetrazine and the second reactive group is a trans-cyclooctene.

6. The method of claim 1, wherein the sgRNA is tagged at a uridine nucleotide in the repeat:anti-repeat region.

7. The method of claim 1, wherein the small molecule suppressor comprises a cell-penetrating peptide or peptide nucleic acid (PNA).

8. The method of claim 1, wherein the suppressor has a molecular weight under about 1200 Da.

9. The method of claim 1, wherein formation of the covalent adduct sterically disrupts the sgRNA/Cas9 interaction.

10. The kit-of-parts of claim 2, wherein the first reactive group is tetrazine and the second reactive group is trans-cyclooctene.

11. The composition of claim 3, wherein the adduct is formed via an inverse-electron-demand Diels-Alder cycloaddition.

12. The method of claim 1, wherein the sgRNA is chemically stabilized using one or more 2′-O-methyl, 2′-Fluoro modifications, or phosphorothioate backbone.

13. The kit-of-parts of claim 2, wherein the first and second components are contained in physically separate compartments of a single package.

14. The method of claim 1, wherein the covalent reaction occurs after a defined time post-transfection.

15. The method of claim 1, wherein the CRISPR suppressor comprises a cell-penetrating peptide, a peptide nucleic acid, a synthetic oligonucleotide analog, or a sterically hindering moiety capable of interfering with sgRNA-Cas9 function.

16. The composition of claim 3, wherein the small molecule CRISPR suppressor comprises a cell-permeable moiety covalently linked to a second bioorthogonal reactive group selected from the group consisting of trans-cyclooctene, cyclooctyne, alkyne, azide, and hydrazine.

17. The composition of claim 16, wherein the second bioorthogonal reactive group is trans-cyclooctene.

18. The composition of claim 16, wherein the small molecule CRISPR suppressor comprises a trans-cyclooctene-modified peptide nucleic acid selected from the group consisting of AAA-PNA-TCO and TTT-PNA-TCO, and is formulated for sequential administration with a tetrazine-modified sgRNA.

19. The method of claim 1, wherein the second composition comprises the composition of claim 16.

20. The method of claim 1, wherein the administration of the second composition is sequentially after introduction of the ribonucleoprotein complex into the cell to enable temporal control of Cas9 activity.
